(19) United States
(12) Patent Application Publication
(10) Pub. No.: US 2023/0248286 A1
(43) Pub. Date: Aug. 10, 2023

(54) CHAIN MAIL MESH DEVICE AND PROCESS FOR REDUCING STRESS

(71) Applicants: CHARLES JOSEPH BRUMLIK, BRANCHBURG, NJ (US); PETER BRUMLIK, BRANCHBURG, NJ (US)

(72) Inventors: CHARLES JOSEPH BRUMLIK, BRANCHBURG, NJ (US); PETER BRUMLIK, BRANCHBURG, NJ (US)

(21) Appl. No.: 18/111,514

(22) Filed: Feb. 17, 2023

Related U.S. Application Data
(63) Continuation-in-part of application No. 17/120,182, filed on Dec. 13, 2020, now Pat. No. 11,602,610.
(60) Provisional application No. 62/947,554, filed on Dec. 13, 2019.

(51) Int. Cl. A61B 5/16; A61B 5/00; A61B 5/11; A61M 21/02

(52) U.S. Cl. CPC A61B 5/165; A61B 5/164; A61B 5/168; A61B 5/4827; A61B 5/1125; A61M 21/02; A61M 2021/0022

(57) ABSTRACT

A device and method of observing mental condition of a user by interacting with an improved swatch of chain mail mesh that comprises interconnecting, polygonal plastic links suitably interconnected to define a sheet having each of the interconnecting, polygonal links having at least one degree of freedom of movement in at least one direction with respect to its neighbouring polygonal links wherein each of the interconnecting, polygonal links, interconnected to at least one neighbouring interconnecting polygonal link, combine to form an operative upper surface and an operative lower surface of the sheet, and wherein at least a portion of at least one of the operative upper surface and the operative lower surface of the sheet generally feels generally smooth with gaps, which produce a calming or pleasing effect when contacted with or moved over, or combinedly contacted with and moved over a human's body portion which being apt to sense of touch. The sheet has a predetermined drape coefficient, nonlinear motion, and is capable of providing relief in stress or pleasure when a human employs the same. The device is a fidget toy, and when used during observation a diagnosis and a therapy tool.

### CROSS REFERENCE TO RELATED APPLICATIONS

The present application claims priority from provisional U.S. patent application 62/947,554, filed on Dec. 13, 2019 and U.S. patent application Ser. No. 17/120,182 filed on Dec. 12, 2020, which are incorporated herein by reference in their entirety.

### FIELD OF THE INVENTION

The present invention relates to a chain mail and, in particular, to a chain mail mesh configured as an anti-anxiety fidget device and tool for reducing and observing anxiety, fidgeting, or stress type symptoms in humans. The present invention also provides a process employing the chain mail mesh for reducing stress or inducing repeated use.

### BACKGROUND

Stress, mental health issues, and related human conditions are omnipresent and inevitable. Stress may be caused due to dissatisfaction in the workplace and personal life. The world health organization classified stress as the health epidemic of the twenty first century. Stress may lead to grave physical health and mental health issues. Physical health issues such as obesity and cardiovascular disease are on the rise. Mental health (a silent killer) has been also linked to cause physical health issues. Mental health issues such as autism spectrum, autistic, attention deficit hyperactivity disorder (ADHD), attention deficit disorder (ADD), Asperger's, anxiety, nervous breakdown, and other mental conditions or negative feelings are also of major concern for all age groups.

Stress, anxiety and related mental states, whatever may be the cause, lead to a reduction in productivity at the workplace and reduces the quality of life. It is estimated that only workplace stress costs about $300 billion annually, in the United States alone, as a result of accidents, absenteeism, and similar reasons. The management of negative mental states also affects every person.

It is of interest to reduce stress type conditions, mental health conditions and negative feelings, to observe their pattern, and to diagnose symptoms or conditions of stress, mental health and negative feelings. Various attempts have been made in the past to develop devices and methods for reducing stress which are especially suited for human use. The methods include various forms of meditation, breathing exercises and the like. However, generally, heretofore, existing devices and methods are somewhat unnatural and do not go a long way in reducing stress, mental health conditions and negative feelings or in the diagnosis of such conditions. This is because generally successful implementation of these methods and devices requires that a person employing such devices and methods to deliberately introduce these in his/her lifestyle and find time for the same and, hence, they are difficult to sustain for long periods of time. Physical diagnostic tools, mental health aids, and fidget toys are also known, the fidget spinner being probably the most popular. These are often banned in schools and workplaces because they can be annoying or distracting to others. Moreover, as far as the applicant knows, these devices have not been used as diagnostic tools.

The present invention shares some structural similarities to chain mail, also called chain mail, maille, or chain maille, which was historically ring mail used for defence against physical attacks. Scale mail was used for similar purposes. Suits and other large areas of chain mail usually combined with a leather undergarment reduced cuts and spread the force of blows from mainly edged weapons. Most of these chain mail pieces were made by combining circular links made of wire formed and soldered or riveted into links connected around other links. While the links may be flat, the links and their connecting links are lying generally perpendicular to each other creating a very rough, non-planar surface. Typical weaves include European (most commonly 4-in-1), Persian, and Japanese. Today, chain mail is still used by butchers and others for cut resistance and other physical protection. They are not sheets, other than some small pieces of such metal circular, perpendicularly-connected links, and may also be used as pot scrubbers (e.g., U.S. D702,009S) that have large circular wire links that are designed to be abrasive and do not feel good against the skin. The weaves typically have more than one connection per loop where each circle connects to at least three other circles in the same open circle. Typical chain mail weaves ranged from 3-in-1 to 6-in-1, may be assembled in a variety of patterns. None of these weaves feel calming against the skin, likely due to significant non-planar rings, the perpendicular orientation of connecting links, sharp edges, lack of flat surfaces, and other design elements which are focused on protection and ease of manufacture with primitive tools.

While not the subject of this invention, one dimensional (1-D) chains are sometimes called chain mail or chains. Typical types of chainmail are ring, flat ring, dragonscale, half Persian, helm, Persian 5 in 1 chain, roundmaille, etc. These may be jewellery approximating historical chain mail elements. Classic jewellery chains, ropes and bracelets are often also made of interconnecting links, but these are very narrow and have a very high aspect ratio. These are designed mainly to bend in limited directions around a neck or wrist. Some have a large tendency to grab hair.

Typical jewellery weave styles which are prettier than chain mail include Cuban, wheat, marine, Singapore, rope, Perfectina, Bismark, curb, Firgaro, criss cross, satellite, bead, sequin, gypsophila, twise, vine, Brillantina, Valentino, Omega, snake, box, byzantine, Jens Pind linkage, spiral rope, etc. These styes are all designed to flex in only one or two dimensions. These styles are typically a single strand of links in one dimension with links that are either generally perpendicular to each connecting link (e.g., box link) or in a traditional chainmail configuration with nonplanar links (e.g., byzantine style). In some configurations, the jewellery can have a rope made of links to bulk in three dimensions. In other configurations, like watch bands there may be a small number of links making up the width of the band. However, these also differ from the present invention in that they only bend in one direction and generally to a limited amount. These bands also are not usually wide enough to provide the intended interplay with the skin. Bike chains and small fidget toys made from a small number of links also differ from the present invention in that they are too small to drape over and contact enough area of skin, only bend in one direction, or do not feel sufficiently smooth. Recently some fidget toys, stress relief toys, and puzzles, advertise themselves as chain mail or chain maille, but they have extremely limited motion. Unlike planar chain mail, these are made into tight cubes, balls, infinity roses, and other highly dense forms that like the very popular fidget spinners are designed to rotate links rather than flex them. Some weaves include Mobius strips, Turkish round, etc. They are not sheets with flattish surfaces, do not bend or drape much, and typically have sharp or protruding corners. NASA, medical device companies, and others are designing new industrial fabrics and chain mail analogues. These are designed for other purposes such as stents, strength to weight ratio, pan scouring, etc. While they can provide visually pleasing aesthetics, none of these forms drape over any appreciable surface of skin and are not designed for comfortable interplay with large areas of skin. In fact, they are typically fairly abrasive and uncomfortable to physically play with for extensive times. Of the more touchable types, the swatch size, weight, and links are not designed for the purpose of repeated hand motion and resulting skin contact.

While wire links containing a single fully open planar loop are not part of this invention, even the types of chain mail and prior uses of interconnected at least partially rigid links with high flexibility between links are not designed for the intended purpose of this invention, and while some may inadvertently approximate some of this invention's advantages, they do not maximize the targeted physiological and psychological effects induced by the use of this invention. They also generally do not minimize the negative effects of historical chain mail, including ring mail and scale mail, such as but not limited to vertical protrusions, pinching, grabbing hair, smell, skin abrasion, excessive thickness, limited drape, limited flexibility, limited range of motion, and limited contact area with the skin. Therefore, there is a need for developing simple, inexpensive ways to observe, diagnose and reduce stress at the workplace and/or in personal life. There is also a need to observe, diagnose and reduce symptoms in mental conditions and suffering.

### SUMMARY OF THE INVENTION

According to an aspect of the present invention a specific type and size chain mail link is provided with a specific size of swatch or mesh and with specific modes of motion. Another aspect of the present invention relates to a process for reducing stress type symptoms or inducing repeated use of the chain mail mesh in humans or providing enjoyment by repeatedly employing the chain mail mesh disclosed herein. Stress or anxiety reduction, or indication or both may be accomplished by the impulsive repeated playing with the swatch of the present invention. The present invention uses some design elements of the chain mail and jewellery and industrial fabrics, adjusting link and swatch size within a narrower range, designing link shapes such as but not limited to triangular or hexagonal with highly rounded corners in 3D and in a hexagonal array, reducing or eliminating sharper edges, so as to improve the desired effects while a hand interacts with the chain mail while reducing the undesired effects such as more angular protrusions during bending, excessive draping beyond hand contact, etc.

According to an aspect of the present invention, a method of utilizing a device to diagnose, observe, or modify a mental condition is provided which comprises: contacting the device with a skin area of the human, wherein the device comprises: a flexible swatch comprising a plurality of discrete, flexibly connected interconnecting links, wherein the swatch forms a sheet having a maximum at rest XY area from 80 cm² to 250 cm²,

wherein the swatch has a weight between 50 grams to 200 grams,
wherein the swatch has a thickness (MT) within 4 mm to 6 mm,
wherein the swatch when extended to its maximum dimensions has a volumetric density of 33-60 vol % as compared to a solid cube of the same maximum dimensions, wherein the swatch thickness does not increase by more than 50% when the swatch is draped over a cylinder with a diameter of at least 10 cm, wherein the swatch comprises 40 to 100 interconnecting links, wherein the links comprise at least 60 vol % plastic and have a material density of 0.5 g/cm³ to 1.9 g/cm³,
wherein the links can move with relation to each other,
wherein the swatch does not materially abrade the skin when moved over the skin solely under its own weight.

The method may further comprise first placing the device over a skin area of the human and moving the device over the skin area of the human under solely its own weight while at least one portion of the device is in contact with the skin area of the human.

In an embodiment, the swatch may form a sheet having a maximum at rest XY area from 100 cm² to 200 cm², wherein the links are flexibly connected in a pattern to form the swatch that allows the swatch to flex in 6 directions relative to an at-rest state in the X-Y plane, wherein the swatch has a weight between 50 grams to 100 grams, and wherein the volumetric density is 40-55 vol % as compared to a solid cube of the same maximum dimensions, the material density of each link is 1.0 to 1.5 g/cm3, the profile diameter of each link varies between 2.5 mm and 3.5 mm, and the sheet thickness (MT) does not increase by more than 50% when the sheet is bent with a radius of curvature of at least 5 cm.

The method may comprise observing the use of the device by the human as a tool for diagnosis or treatment of the mental condition or of symptoms of the mental condition. The observing may be performed electronically via a camera recording the use of the device by the human. Alternatively, the use of the device by the human may be observed by a medical or mental health professional.

The mental condition, indicator, or symptoms of the mental condition may comprise at least one of fidgeting, stress, post-traumatic stress disorder (PTSD), trauma, attention deficit disorder (ADD), attention deficit hyperactivity disorder (ADHD), autism spectrum, distraction, annoyance, lying, general anxiety, situational anxiety, depression, motor function, touch sensitivity, isolating psychiatric conditions, trauma, and the like.

The mental condition or symptoms of the mental condition may comprise lying (lie detection), therapy, preparing for therapy, coming down from psychotherapy efforts to change thinking, preparing mental state, diagnosing stress, diagnosing sources of anxiety, diagnosing depression, treating stress, treating anxiety, treating depression, treating effects of trauma, and the like.

In an embodiment, the method comprises observing the use of the device by the human as a tool for

i) diagnosis of mental condition or symptoms of mental condition including lying, stress, anxiety, depression and the like,

ii) diagnosis of at least one reason for the diagnosed lying, stress, anxiety, depression and the like,

iii) for treating the mental condition or symptoms of the mental condition;

iv) determining the efficacy of the treatment followed by the human for the therapy of the diagnosed mental condition or symptoms of the mental condition; and

v) identifying an indicator of stress, anxiety or other mental condition

The method according to claim 5, wherein the diagnosis (i) or (ii) or the determination of the efficacy of the treatment (iv) includes observing use of the device by the human at at least one predetermined interval following the treatment.

The diagnosis (i) or (ii) or the determination of the efficacy of the treatments (iv) may include asking the human to repeat a pattern of use of the device, or merely handle the device without specific direction, during an observation session having a predetermined observation time, recording a video via a camera of the use of the device by the human, and analyzing the video. The analysis may include collecting usage data such as the number of the repetitions performed by the human within the observation time, the particular movement pattern of the device over the skin area of the user, the facial expression of the user and the like. The video and the analyzed data may be reviewed by an AI system and/or an actual professional including a medical doctor, a psychiatrist, a physiotherapist and the like.

The method may comprise a plurality of observation sessions for diagnosis (i) or (ii), or for determining the efficacy of the other treatments (iv).

The method may comprise a plurality of treating sessions for treating the at least one diagnosed stress, anxiety or depression via us e of the device by the human.

The method may comprise, during at least some of the treating sessions, a professional including a medical doctor, a psychiatrist, a physiotherapist and the like observing the use of the device by the human and collecting use parameters including the number of repetitions performed by the human of a particular pattern of use prescribed by the professional during an observation time wherein the observation time may be the same or less than a treating time.

The method according to claim 1, wherein the method comprises placing the device on a body portion of the human which is apt to sense of touch, and moving the device over the body portion, wherein said device intermittently contacts skin areas near nerve endings of said body portion thereby providing a calming effect to the human.

The method may include the human user moving the chain mail mesh from hand to hand or with a single hand. The method may comprise the human holding the chain mail mesh with one hand, contacting the chain mail mesh with a skin portion of the other hand, and moving the chain mail mesh over a skin portion of the other hand such that at least one of a lower operative surface and an upper operative surface stimulates said nerve endings of said skin portion thereby inducing the human to increase rate or frequency or both of motion of the chain mail, and observing such visually, audibly or both.

The device may comprise the format of a keychain, attachment to a sales placard with branding and UPC code, or similar detachable pendant.

In an embodiment, the method comprises placing the device on a body portion of the human which is apt to sense of touch, moving the device over the body portion, a professional including a medical doctor, a psychiatrist, a physiotherapist and the like observing the use of the device by the human to collect use parameters including the frequency of the use, and determining based on the collected use parameters at least one of i) whether the human is lying, or is suffering from at least one of stress, anxiety, and depression, ii) whether a prescribed treatment for the at least one of the stress, the anxiety and the depression is reducing the stress, the anxiety or the depression, iii) whether a repeated use of the device by the human reduces at least one of the stress, anxiety, and depression of the human, and wherein the device intermittently contacts skin areas near nerve endings of said body portion thereby providing a calming effect to the human, wherein the X-Y shape of the swatch approximates a shape between a rectangle and a circle, wherein the shape of the link comprises both curved and flattened surfaces, wherein the shape of the link is adapted to provide a slightly rough external surface, wherein each link has a maximum XY area from 1 cm2 to 5 cm2, a maximum Z height from 5 mm to 10 mm, and an average post thickness from 2 mm to 4 mm, wherein each link has a ratio of maximum X:Y dimensions of 1:1 to 1:1.5, wherein the swatch comprises essentially similar links, wherein every link is attached to 2 to 6 other links, wherein the majority of links are attached to 6 other links, wherein the links comprise plastic and the swatch does not have any touchable protrusions that are sharp enough to reduce mental state of the human.

The swatch may include an operative lower surface and an operative upper surface which are substantially identical, and may have a repeating pattern with a changeable gap between the links, the gap being from 0.1 mm to 0.4 mm when at rest and a maximum of 0.2 mm when neighbouring links contact each other.

According to an aspect of the present invention a device is provided which is suitable for improving a mental state or condition or of symptoms of a mental state or condition in a human, the device comprising a chain mail mesh comprising a plurality of interconnecting polygonal links suitably interconnected to define a sheet having a length (ML), a breadth (MB), a thickness (MT) and weight (MW), each of said interconnecting polygonal links having at least one degree of freedom of movement in at least one direction with respect to its neighbouring polygonal links, each of said interconnecting polygonal links having a length (LL), a breadth (LB), a thickness (LT), and weight (LW), wherein each of said interconnecting polygonal links is interconnected to at least one neighbouring interconnecting polygonal link, and

wherein said plurality of interconnecting polygonal links combine to form an operative upper surface and an operative lower surface of said sheet, and
wherein at least a portion of at least one of said operative upper surface and said operative lower surface of said sheet is sufficiently flat and smooth and of a repeating pattern of the links such than when moved over a body part of the human being apt to sense of touch, induces repeated use or produces a calming effect.

A device for the diagnosis and treatment of stress, the device comprising a sheet comprising a chain mail mesh,

wherein the chain mail mesh comprises a continuous rod or pipe of a plastic material,
wherein the rod or pipe form three to six valleys and three to six hills spaced apart from each other at a regular interval in a closed loop configuration
wherein each valley is followed by a hill and each hill is followed by a valley,
wherein between each valley and an adjacent hill the rod or pipe forms a slanted intermediate element connecting the two together in a continuous manner. In one embodiment, there are four to six valleys and hills.

According to an embodiment, the device comprises a sheet having a generally triangular shape of three equal sides having a length of 50 mm to 250 mm, wherein a thickness also referred to as a height defined as a vertical distance from a bottom surface of a valley from a top surface of a hill of 3.0 mm to 8.0 mm, and wherein the diameter the rod or pipe is between 2.5 mm and 3.5 mm.

The device may preferably comprise a rod or essentially solid pipe having a circular, or oval cross-section of a substantially constant diameter. The closed loop configuration may preferably be circular, oval, or triangular. Each hill and each valley may have an external surface defined as the surface that comes into contact with the skin of the human when the device is used by the human as a fidget device as a toy or for the diagnosis or control of a mental condition such as stress and the like having an RMS smoothness of 20 to 600, preferably 100 to 600, and more preferably 200 to 600. The external surface may in an embodiment include at least one central portion that is substantially flat.

In the embodiment of FIG. 6, each link may be coupled to three other links except for the outmost links each of which may be coupled to only two other links. When swatch is extended on a flat surface, a maximum gap between two adjacent interconnected links may range from 0.1 mm to 0.4 mm. The device may have a weight of 50 to 200 grams.

In one aspect, the present invention provides a specific chain mail mesh for reducing stress in humans and improving a general feeling of wellbeing when humans interact with the chain mail mesh. Unlike most chain mail designed for physical protection or abrasive scrubbing, the specification of the present invention chain mail mesh does not provide any material physical protection. The specification of the present invention chain mail mesh is designed to provide stress reduction and mental relief. The specification of the present invention chain mail mesh is designed not to materially abrade the skin and to balance stimulation with relaxation effects. The chain mail mesh, in accordance with the present invention, comprises a plurality of interconnecting, polygonal links suitably interconnected to define a sheet, when laid on a flat surface. The sheet is characterized by having a length (ML), a breadth (MB), a thickness (MT), an overall bumpiness or periodicity of links and gaps within the sheet, weight (MW), and modes of motion during movement, which are suitably chosen to enhance the stress reduction aspect. Each of the interconnecting, polygonal links have at least one degree of freedom of movement in three directions or three dimensions, when not at the edge of the chain mail mesh, with respect to its neighbouring polygonal links. Each link is characterized by a length (LL), a breadth (LB), a thickness (LT), an average profile diameter (LD), and a weight (LW) and have an operative upper surface, an operative lower surface, and one or more substantially rounded edges. The operative upper surface, the operative lower surface and other surfaces of the links that become exposed and can rub against the skin during normal use, preferably have one or more substantially rounded edges. The average profile diameter (LD) may have an overall approximated profile shape selected from the group consisting of elliptical, flat, rounded, convex, and any combinations thereof. The link cross-sectional profile and the average profile diameter (LD) is measured perpendicularly to the center line of the line and preferably changes along the closed loop of that centerline. In an embodiment, the interconnecting, polygonal links are interconnected directly to the at least three neighbouring interconnecting, polygonal links, directly or via an intermediate connecting member. For interconnecting, polygonal links at the edge of the chain mail mesh, the interconnecting, polygonal links may only be connected to two intermediate connecting members. The contact area of the swatch, i.e., the area of the interconnected links which come into contact with the skin of a user, for example, the skin of a hand or fingers is flat or has a flat surface. Flat means geometrically flat or approximately flat when compared to the rest of the link profile or the swatch profile. While not flexible in the manner of the present invention, for explanatory purposes, a Cuban link us in jewellery typically has a flat top and flat bottom and rounded other surfaces. An individual link not connected to other link is not measured because it may have side or internal corners or surfaces not intended to significantly contact the skin in the final swatch. Preferred measurement is percent external surface area of the swatch or link, as measured by the percentage of link in the outer 10% of the MT that may be felt by an adult hand when the swatch is at rest or draped over a hand.

In an embodiment, the sheet may be largely circular or octagonal in shape and may have about 25 to 250 interconnecting, preferably 50-100, polygonal links and allows the swatch to drape in more than two directions. In a preferred embodiment, there are no links that are attached to only one other link.

The sheet is configured to drape when contacted with or moved over, or combinedly contacted with and moved over a human's body portion, preferably over one or both hands, and substantially conforms to the shape of the body portion, wherein the operative lower surface or the operative upper surface, or preferably both lower and upper operative surfaces of the sheet in contact with the body portion provides a calming, mood, or enjoyment effect when physically interacting with a hand or other human's body portion. The thickness (MT) of the sheet, when the sheet is in draped configuration, may be up to 50% larger, or alternatively up to 30% larger, than the thickness (MT) of the sheet when the sheet is placed on a substantially flat surface, where under at rest (MT) is measured. However, excessive raising of corners during bending, such as with unrounded square links, are preferably minimized as they can induce discomfort. The sheet has a predetermined drape coefficient. In an embodiment, the drape coefficient is the same along at least two axes of the sheet. In another embodiment, the drape coefficient is different along the length (ML) and the breadth (MB) of the sheet, (also, referred to as the width of the sheet. The thickness (MT) should not be too large because it can interfere with the flexibility of the relatively small sheet and, more importantly, does not feel as comforting as the preferred thickness (MT) of 4-10 mm, more preferably 4-6 mm for links comprising plastic. A smaller thickness does not feel as comforting because it has insufficient weight and has a feeling too similar to a garment textile.

In an embodiment, in addition to the length, the breadth, and the thickness design parameters of the sheet, an acoustic spectrum may be generated when the sheet is displaced by a user. Typically, the acoustic spectrum may comprise one or more audible frequencies, preferably in the range of 20 Hz to 2,000 Hz, more preferably predominantly within 500 Hz to 1,500 Hz. The sheet may be tuned to a certain frequency band or waveform to provide a user with a relaxing and calming sound by suitably choosing the material of make, the dimensions, the gap, the surface, and the structure of the interconnecting, polygonal links and, hence, that of the sheet when in motion during a typical hand play.

Further, in the chain mail mesh, the interconnecting, polygonal links are interconnected to three to six, and preferably three, neighbouring interconnecting polygonal links, which combine to form an operative upper surface and an operative lower surface of the sheet. At least 30%, more preferably at least 50% of the operative upper surface and an operative lower surface is a contact surface that may contact the skin during normal use, wherein either of the operative upper surface or the operative lower surface or preferably both are configured to contact with at least a body part of a human, and provide a calming effect thereto. However, links positioned at corner or edges of the swatch may connect to at least two, neighbouring interconnecting polygonal links. More specifically, at least a portion of at least one of the operative upper surface and the exposed link surfaces in the operative lower surface of the sheet generally feels smooth. In a preferred embodiment smooth is defined herein as an RMS surface finish between 20 and 600. RMS is the root mean square average of the profile height deviations from the mean line as described in AME B46.1. In a preferred embodiment, each of the interconnecting, polygonal links are interconnected to three neighbouring interconnecting polygonal links. The combination of this rigid pleasant surface with intermittent air gaps and nonlinear motion produces a calming or enjoyment effect when contacted with or moved over, or combinedly contacted with and moved over a human's body portion which is apt to the sense of touch. The interconnecting, polygonal links within the sheet are spaced apart with a gap formed therebetween, which configures a loose hinge like configuration between the interconnecting, polygonal links. Further, at least a portion of the sheet which is bendable in at least one and preferably in both Z directions selected from an operative upward direction of the operative upper surface of the sheet, an operative downward direction of the operative lower surface of the sheet and a combination thereof. The gap along with the loose hinge configuration provide the sheet with bendability that permits the sheet to drape when placed on a suitable support, very much like a piece of fabric, though in a more nonlinear motion mechanism. In addition to the bendability, at least a portion of the sheet may be skewable in at least one direction substantially parallel to a plane of the sheet. In an embodiment, the gap is in the range of 0.1 mm to 7.0 mm, preferably 0.3 mm to 4.0 mm, and, more preferably 1.5 mm to 3.0 mm. For example, in a 10 cm×10 cm swatch with 0.7 cm×0.7 cm links, the links are made up of connector members with an average thickness or diameter of 2.0 mm, and a gap of 2.5 mm. The XY area of the gap is preferably less than the XY area of the solid link, as measured by percent light transmission through a swatch when the swatch is in a configuration with its smallest XY area on a light table. In one embodiment the maximum gap while skewed is measured with a caliper. Due to the rigid links and constrained motion, this means that the sheet is not completely flexible mainly due to the rigid links and provides some resistance to drape within its range of motion, in contrast with a typical textile fabric.

In an embodiment, the periphery of the sheet may be terminated by employing polygonal end cap links. The polygonal end cap links are connected to an outer most layer of the interconnecting, polygonal links of the sheet. The number of layers or units of the polygonal end cap links may vary. In an embodiment, the number of layers of the polygonal links may be one. In another embodiment, the number of layers of the polygonal links may be two or more. Preferably the polygonal end cap links may be the same as that of the interconnecting, polygonal links. In one embodiment, the polygonal end cap links may be different from that of the interconnecting, polygonal links, but do not materially limit the drape of a majority of the swatch. An example is a keychain pendant. The structure of the interconnecting, polygonal links is chosen such that the operative upper surface, and/or the operative lower surface of the interconnecting, polygonal links are generally smooth to touch, thereby the operative upper surface and the operative lower surface of the sheet are also bumpy yet otherwise substantially smooth to touch. This is not meant to mean that a user cannot feel a surface pattern on the link surface. In an embodiment, the interconnecting, polygonal links comprises at least three lower horizontal connector members, which define the operative lower surface and at least three upper horizontal connector members, which define the operative upper surface, wherein the at least three lower horizontal connector members being connected to the at least three upper horizontal connector members at corner posts to define an incessant link. In a preferred embodiment, the connecting members comprise curves between horizontal and vertical.

The provision of the lower transverse intermediate connector member and upper transverse intermediate connector member further enhances the smoothness of the operative upper and the lower surfaces of the interconnecting, polygonal links and that of the sheet and at the same time provides strength to the interconnecting, polygonal links. In the preferred embodiment all members comprise the same type of link.

In accordance with the present invention, the polygonal links, the optional loops, and the optional polygonal end cap links may be each independently manufactured from a polymer and optionally also a relatively smaller volume percent of a material selected from the group consisting of plastic, elastomer, thermoplastic, composites metal, ceramic, glass, mica, fillers, foaming agent, glitter, pigment, processing aid, and any combinations thereof. In a preferred embodiment the links comprise at least 60 vol % polymer if used with a filler or coating. In another embodiment, the links comprise at least 90 vol % polymer. Plastics may comprise polymers including thermosetting polymers, UV cured resins, thermoplastics, polyolefin homopolymers, polyolefin copolymers, polymer blends, and the like. In an embodiment, the links may be covered with a coating covering the surface of the links. For example, the coating may be a metal coating, ink, dye, fragrance, oil, or a paint coating. The paint coating may be, for example, a polymer coating. The metal may include metals and metal alloys. In an embodiment the metal may be an aluminium alloy. Various non-precious metals may be coated using any suitable coating method such as, for example, electroplating. Ceramics, metal composites, ceramic composites, polymer-inorganic composites, or polymers may be metal coated or metal filled. In one embodiment, gold, titanium nitride, or silver or other decorative or protective coating may be employed.

The choice of the material depends on the drape of the sheet to be achieved, the body portion over which the sheet is used, and the weight of the sheet to be achieved. In an embodiment, the interconnecting, polygonal links and the polygonal end cap links are each manufactured from plastic in the same production machine. In an embodiment, the interconnecting, polygonal links and the optionally polygonal end cap links are each co-dependently molded or 3D printed from plastic.

In an embodiment, the maximum length (ML) and the maximum breadth (MB) of the sheet are each independently in the range of 50 mm to 250 mm, and the thickness (MT) of the sheet is in the range of 3 mm to 8 mm, when the sheet is placed on a substantially flat surface, whereas the length (LL), and the breadth (LB) of the interconnecting, polygonal links are independently in the range of 10 mm to 20 mm. Maximum is used here because a preferred embodiment is not a rectangle. In a preferred embodiment, the X:Y ratio of the sheet may be from 1:1 to 1:3, preferably approximately 1:1 for an approximated square or circle of the same area as the swatch. It is noted that X and Y, or X, Y are used herein to refer to the plane and sheet length and width of a swatch, respectively, when at rest. X is also referred to as length (ML), and Y is also referred to as breadth (MB).

In accordance with an embodiment, the interconnecting, polygonal links may have a connection form that is at least one selected from the group consisting of a three-sided polygonal link, a four-sided polygonal link, a five-sided polygonal link, and a six-sided polygonal link. The preferred connection form is to have loops on three to six, preferably three, sides of the link in the X-Y plane. The overall visual appearance of the link can differ from the connection pattern of the swatch. In an embodiment, the interconnecting, polygonal links, while having void spaces, may have at least one overall, approximate visual shape selected from the group consisting of a circle, triangle, a square, a rectangle, a pentagon, a hexagon a higher order polygon, versions of these with rounded corners, and combinations thereof. In another embodiment, the interconnecting, polygonal links are triangular in shape with rounded corners when the top surface or bottom surface is looked at from the Z direction. In another embodiment, the interconnecting, polygonal links are squat, generally cylindrical in shape with optional X-Y protrusions or pillars at least partially in the Z-plane. In an embodiment, the interconnecting, polygonal links frame out a non-square shape in the X-Y plane, a flat top and bottom, same structure as the other polygonal links, and a (ML) and (MB) generally similar and in the range of 10 mm to 18 mm and (MT) in the range of 3 to 7 mm and are made of thermoplastic or 3D printed polymer-based material.

The chain mail mesh, the interconnecting, polygonal links, and the end cap polygonal links may be each independently manufactured by any known technique including, but not limited to, 3D printing, injection molding, casting, polymer wire forming, snap fitting and combinations thereof.

In another aspect, the present invention provides a method of reducing stress or otherwise improving mental state in humans by contacting with and moving over, or combinedly contacting with and moving over a human's body portion which being apt to sense of touch, the chain mail mesh as described hereinabove, wherein the chain mail mesh intermittently contacts near nerve endings of the skin body portion thereby provide a calming or other positive effect thereto.

In still another aspect, the present invention provides a process for reducing stress or otherwise improving mental state in humans, the process comprising the steps, including holding the chain mail mesh, in one or both hands of a human, contacting the chain mail mesh with a body portion of the human and/or moving the chain mail mesh over the hand or other body portion of the human such that the lower operative surface and the upper operative surface contacts with skin or near nerve endings of the body portion and thereby provide a calming effect thereto, and repeating the above steps more than once to reduce the stress in humans. The weight of the swatch may also provide relief when at rest.

The body portion is the general skin and nerve ending region, which is approximately and partially contacted with and over which the chain mail mesh is moved over time. Preferably the body portion is at least one selected from the group consisting of hand, hands, wrist, head, forehead, shoulder, back, chest, neck, finger, calf, knee, thigh, hip, waist, foot, heel, ankle, buttock, leg, skin, and stomach and is not limited to these movement method incudes physical interaction of a subject with the swatch, and optionally visual and auditory senses. The preferred body portion is fingers, hand, and portions of the hand. Because the swatch of links provides feelable solid and air combinations and textures that at least nonlinearly change with motion, the resulting effect on the subject differs from that of typical textiles. These intermittent skin contact physical senses then lead to mental and central nervous system (CNS) effects. The size, link texture, weight, and swatch surface that may contact the skin, all are carefully selected to reduce negative effects such as scouring of the skin, and to increase positive effects such as repeated stimulation, or other stimulation not creating material, lasting harm to the user. The sound during use and pleasing or familiar look may also contribute to this effect, either directly or as a secondary impulse.

[0027A] Method involving a single person—These resulting effects on the subject may be used to both directly affect the anxiety or other mood of the subject or as a tool by an observer of the person handling the swatch. The present invention may become a secondary stimulus wherein the user develops a connection between holding or playing with the swatch and a positive feeling or at least a reduction of negative feelings, thereby creating an impulse to pick up and optionally play with the swatch. Examples of expected use, can include but are not limited to: relieving stress, relieving anxiety, fidgeting, helping focus, distracting, providing mental comfort, reducing outbursts, and the like.
Method involving use plus observation—The general applications of the method of the present invention that involve more than a single person or observation of any kind including via camera, microphone or both, generally comprise testing, diagnosis, treatment, or combinations. Examples to test, diagnose, or modify psychological states or related behaviours, include but are limited to lie detection, a tool for psychological tests. The rate of motion and resulting sound are at least sometimes proportional to the level of anxiety, stress or nervousness. The propensity to pick up, play with, or put down the swatch of the present invention may also be indicative of state of mind and changes thereto.
Previous diagnostic psychological tests such as the block design test use physical aids though many of these that do not relate to physical coordination may simulate physical aids using online versions. In this invention, the physical tool is critical to the use of the present method since the physical stimuli and interaction directly interact with the central nervous system (CNS) rather than relying merely on visual input and conversion.
The present invention can also be used as an external indicator or the subject's mood in applications such as psychological therapy, interviews, interrogation, lie detection, and the like. As an example of a therapy aid, the present invention may also be used by a practitioner to help calm their patient as well as to better and faster sense their mood. This can be used to replace or reduce drugs to better observe psychological variables during therapy and observation, to help isolate and differentiate different contributing factors such as how a patient responds upon stress and then how that patient responds during a calming period with the present invention. Visual, sound, motion, or any combination of the device of the present invention or the hand or body motion instigated or exaggerated by its presence may benefit these applications. Examples of expected use, can include but are not limited to: therapy, preparing for therapy, coming down from psychotherapy efforts to change thinking, preparing mental state, lie detection, therapy, preparing for therapy, coming down from psychotherapy efforts to change thinking, preparing mental state, diagnosing stress, diagnosing sources of anxiety, diagnosing depression, treating stress, treating anxiety, treating depression, treating effects of trauma, and the like.
Examples of expected tests using the present invention as an aide or diagnostic, can include but are not limited to: lie detection, fidgeting, stress, PTSD, trauma, ADD, ADHD, autism spectrum, distraction, annoyance, lying, general anxiety, situational anxiety, depression, motor function, touch sensitivity, isolating psychiatric conditions, trauma, and the like.

### BRIEF DESCRIPTION OF THE DRAWINGS

Some embodiments of the presently disclosed invention are illustrated as an example and are not limited by the figures of the accompanying drawing, wherein like references may indicate similar elements and in which:

FIG. 1 illustrates an isometric view of a chain mail mesh of the presently disclosed invention, wherein the chain mail mesh is in the form of a sheet, with identical square-shaped interconnecting, polygonal links;

FIG. 2A illustrates a side view of an interconnecting polygonal link in accordance with an embodiment of the present invention;

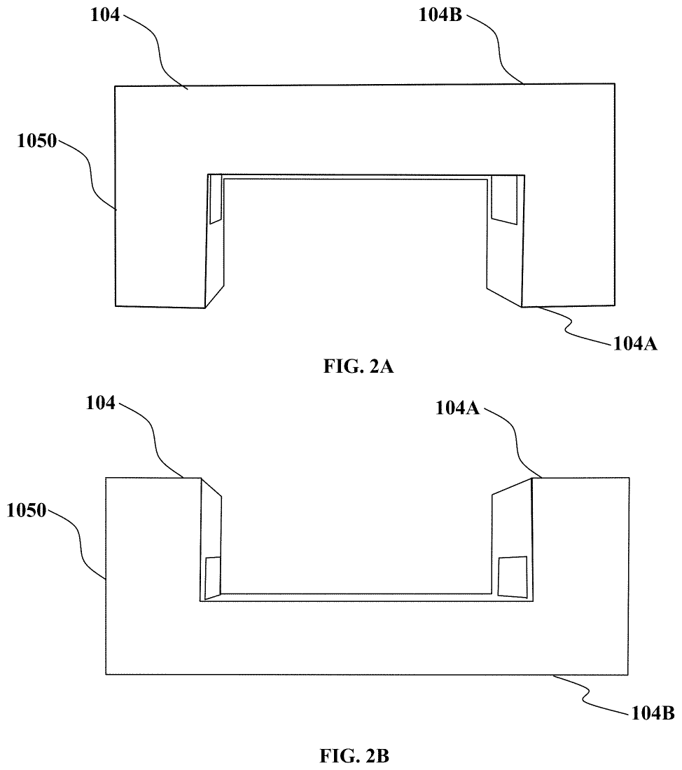

FIG. 2B illustrates another side view of the interconnecting polygonal link of FIG. 2A;

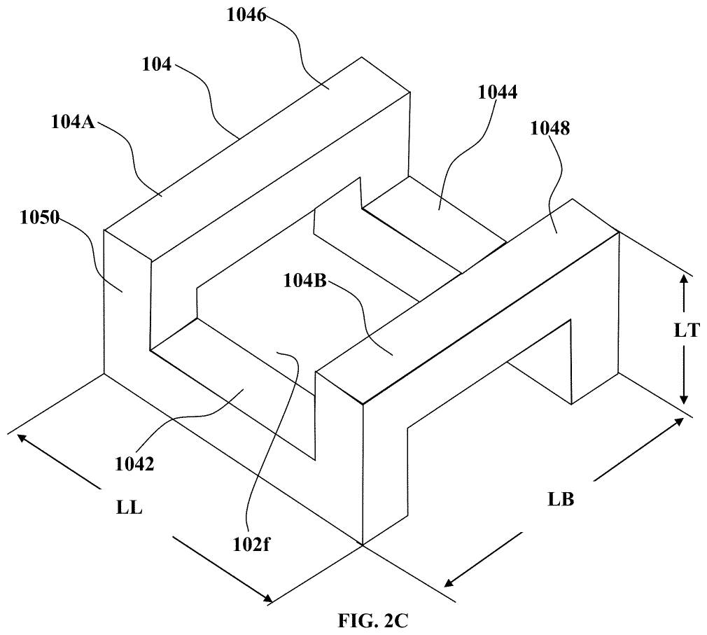

FIG. 2C illustrates a perspective view of the interconnecting polygonal link of FIG. 2A; FIG. 3A illustrates a perspective view of a pair of interconnecting, polygonal links of FIG. 2A, wherein two interconnecting, polygonal links (having a square shape) are connected together;

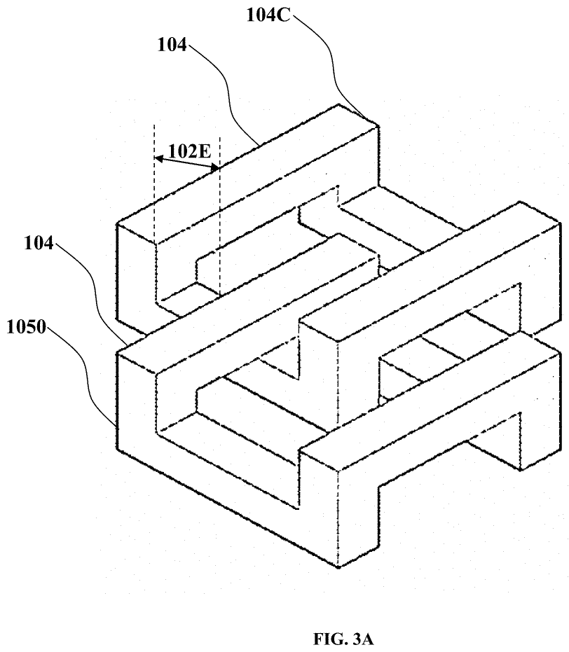

FIG. 3B illustrates a top view of the pair of the interconnecting, polygonal links of FIG. 3A;

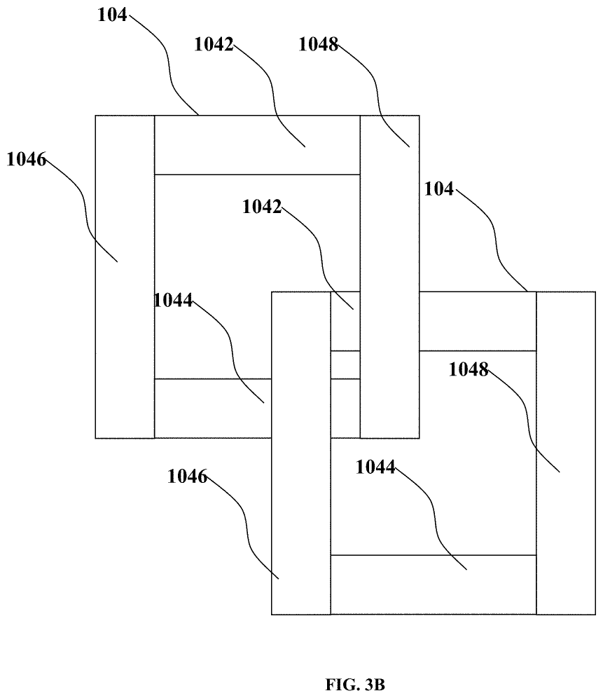

FIG. 4A illustrates an isometric view of an interconnecting polygonal link in accordance with another embodiment of the present invention;

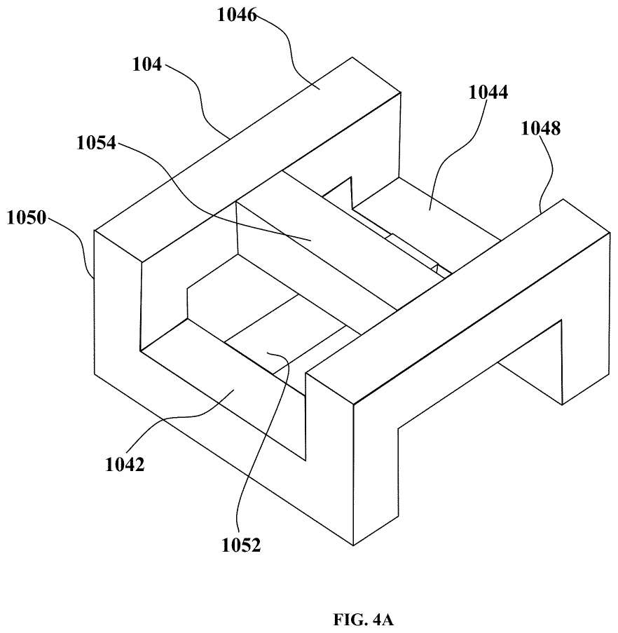

FIG. 4B illustrates a top view of a pair of interconnecting, polygonal links of FIG. 4A, wherein two interconnecting, polygonal links (having a square shape) are connected together; and

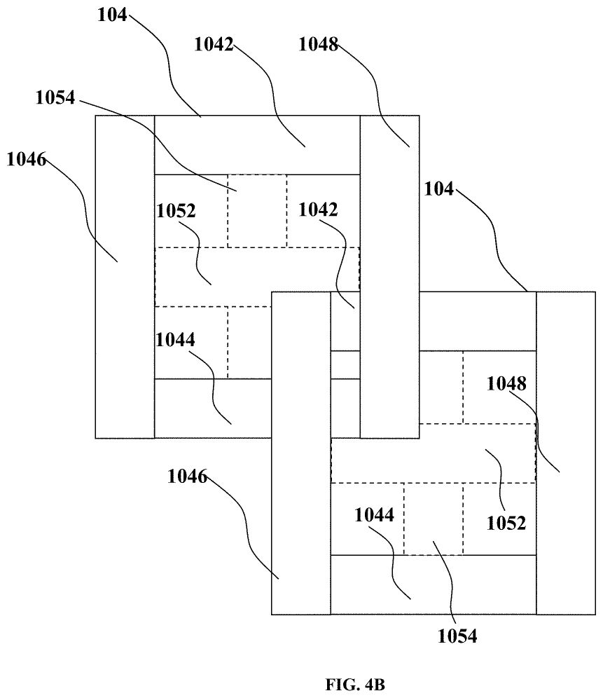

FIG. 5 illustrates a conventional chain mail mesh having ring-shaped interconnecting links and made of metal.

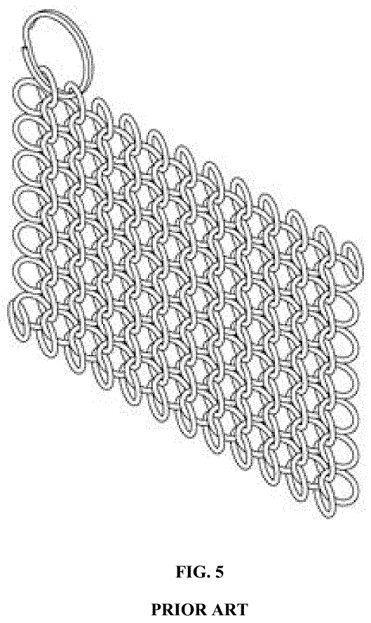

FIG. 6 illustrates a perspective view of a chain mail mesh according to an embodiment of the present invention, the chain mail mesh comprising a plurality of links wherein each link is coupled to three other links except some of the outermost links each of which is coupled to two other links.

FIG. 6A illustrates a perspective view of the link employed in the embodiment of FIG. 6.

FIG. 6B illustrates two of the links of the embodiment of FIG. 6 coupled together.

FIG. 6C illustrates a link coupled to three other links according to the embodiment of FIG. 6.

### LIST OF NUMERALS

100, 600—Chain mail mesh
102, 602—Sheet
102A, 602A—Operative upper surface of the sheet 102
102B, 602B—Operative lower surface of the sheet 102
102C—Operative upward direction
102D—Operative downward direction
102E, 604E—Gap between two interconnecting, polygonal links
104, 604—Interconnecting links
104A—Operative upper surface of the interconnecting, polygonal links
104B—Operative lower surface of the interconnecting, polygonal links
104C—Smooth edges
104D—Outermost layer of the interconnecting, polygonal links 104
604V—Valley region of an interconnecting link
604H—Hill region of an interconnecting link
604C—Central region of a hill or valley of an interconnecting link
604Z—Profile diameter
604S—Side or intermediate section of link 604
1042—Lower horizontal connector member (first)
1044—Lower horizontal connector member (second)
1046—Upper horizontal connector member (first)
1048—Upper horizontal connector member (second)
1050—Corner posts
1052—Lower transverse intermediate connector member
1054—Upper transverse intermediate connector member
106—polygonal end cap links
ML—Length of the sheet 102
MB—Breadth of the sheet 102
MT—Thickness of the sheet 102
LL—Length of the interconnecting polygonal link 104
LB—Breadth of the interconnecting polygonal link 104

### LIST OF VARIABLES

I—Length (ML) of the sheet 102
II—Breadth (MB) of the sheet 102
III—Thickness (MT) of the sheet 102
IV—Length (LL) of the interconnecting polygonal link 104
V—Breadth (LB) of the interconnecting polygonal link 104
VI—Thickness (LT) of the interconnecting polygonal link 104
VII—Colour of the sheet 102
VIII—Colour of the interconnecting, polygonal links 104
IX—Colour of the polygonal end cap link 106
X—Material of make of the interconnecting polygonal link 104
XI—Material of make of the polygonal end cap link 106
XII—Gap (102E) between two interconnecting, polygonal links 104
XIII—Drape coefficient of the sheet 102
XIV—Radius of curvature of the sheet 102
XV—Weight (MW) of the sheet 102
XVI—Weight (LW) of the interconnecting, polygonal links 104
XVII—Number of sides of the interconnecting, polygonal links 104
XVIII—Shape of the interconnecting, polygonal links 104
XIX—Number of sides of the polygonal end cap link 106
XX—Shape of the polygonal end cap link 106
XXI—Acoustic spectrum frequency band emitted by the chain mail mesh 100
XXII—Operative upper or operative lower surface area of the sheet 102
XXIII—Operative upper or operative lower surface area of the interconnecting, polygonal links 104
XXIV Length of the intermediate connecting member
XXV Breadth of the intermediate connecting member
XXVI Thickness of the intermediate connecting member
XXVII Weight of the intermediate connecting member
XXVIII Material of make of the intermediate connecting member
XXIX Shape of the intermediate connecting member

### DETAILED DESCRIPTION OF THE INVENTION

All technical terms and scientific expressions used in the present invention have the same meaning as understood by a person skilled in the art to which the present invention belongs, unless and otherwise specified. As used in the present specification and the claims, the singular forms “a”, “an” and “the” include plural references unless the context clearly dictates otherwise.

The term “comprising” as used in the present specification and the claims may be understood to mean that the list following is non-exhaustive and may or may not include any other extra suitable things, for instance, one or more additional feature(s), part(s) and/or constituent(s) as applicable.

Further, the terms “about” and “approximately” used in combination with ranges of sizes of parts, particles, compositions of mixtures, and/or any other physical properties or characteristics, are meant to include small variations that may occur in the upper and/or lower limits of the ranges of sizes.

Swatch or Chain mail or chain mail mesh is defined herein to mean a fabric of interconnecting rigid links, wherein the links are large enough to be individually felt by human fingers or any body parts and their connection flexible and loose enough to drape the fabric over a body part of a human, for example, a hand of a human. Rigid link means the link has a stiffness or flexural modulus of plastic, preferably at least 1.5 GPa, as measured by ASTM D790 and ISO 178 bend test method but less than mild steel.

Stress as this term is employed herein may also include other human conditions, symptoms, and manifestations of mind, body or both that cause a human to feel less than happy or calm. Representative examples of such moods could include anxiety, fear, depression, ADHD, perseveration, autism spectrum, nervousness, lying, awkwardness, etc.

Smooth surface or edge refers to the texture of a surface or edge which feels generally smooth to the human touch and is substantially free of sharp edges or profiles that could materially damage the skin during normal intended use of the present invention. However, one embodiment comprises surface roughness on the link surface and line edges in the transition between curved and flat surfaces.

Loose hinge or loose loop as used herein means a coupling or a connection between two adjacent interconnecting, polygonal links and/or an interconnecting polygonal link and a polygonal end cap link and/or between an interconnecting polygonal link and an intermediate connecting member, wherein the coupling or connection is similar to a hinge in allowing constrained motion, but is loose thereby enabling relatively free movement of the interconnecting, polygonal links, polygonal end cap links and intermediate connecting member as compared to conventional hinges. Free movement means both looseness or rattle, and movement outside of the single rotational direction. Loop is a functional description of this loose connection means. As a hinge has loops and a pin, the links have constraining closed loops or partially constrained loop sections that constrain loops of portions of neighbouring interconnecting, polygonal links. A loop may also comprise integral structural parts of the interconnecting, polygonal links.

Neighbouring interconnecting, polygonal links may include an immediate neighbouring interconnecting polygonal link or may also include a connecting link between such interconnecting polygonal links.

Skewable as this term is used herein means that the sheet and/or the chain mail mesh may be at least slightly distorted at least either lengthwise or breadthwise or both by moving the interconnecting, polygonal links and/or the polygonal end cap links and/or the intermediate connecting members.

The at rest state or XY area of the swatch refers to the area defined by the swatch perimeter in the X-Y plane when the swatch is placed on a flat, hard surface on the X-Y plane, gently pulled out to its maximum XY area, and released to rest on the flat, hard surface.

Link looseness refers to the play between the loop and the adjoining link. For example, loops have at least one inner dimension that is larger than the connecting link part in that loop to allow some play in the motion between the link with the loop and the connecting link. This play may often allow features such as fast motion, looseness in motion, noise of rattles or j angles, as compared to a limited motion device such as a door hinge.

Resistance to motion may limit the motion of the neighbouring links and of the swatch in aspects such as direction of motion, limit of motion, speed, acceleration, friction, and the like. It is mainly controlled by link location, link shape, link looseness, and collision between adjacent link that may constrain maximum range motion. In a preferred embodiment, link motion is not materially slowed.

Drapability also referred to herein as drape is the ability of the swatch to significantly flex such that at least a majority, and preferably all, of the XY edges of a swatch bend at least over itself when placed over a cylinder or rod with a diameter approximately equal to the average diameter of the largest link in that swatch. High drape is desired unlike chain mail and other armour where high drape reduces physical protection.

The drape coefficient of composite textiles is generally more difficult to measure than traditional textiles. An objective method for the determination of fabric samples mechanical properties is known as the “jutting strip method” or “cantilever method” which has been developed by Pierce in 1930 and is included in the FAST system (Fabric Assurance by Simply Testing). The bending property, making a part of the overall mechanical characterization of the swatch may be correlated to a drapability. See, Smart Textiles for In Situ Monitoring of Composites, Vladan Koncar, Woodhead Publishing, Oct. 29, 2018—Technology & Engineering, p. 218. For simplicity, this invention measures both drape and drape coefficient as the minimum deflection in the Z direction of a swatch in the X-Y plane. A plastic swatch may have a smaller radius of drape, where drape will not substantially differ over a wire, or measurement circle of up to 0.5 cm because it typically will have larger links. A metal swatch due to mechanical strength can have smaller links and smaller connecting means than plastic and, therefore, would have a larger drape because while still a composite, it can move in a manner closer to that of a traditional non-composite textile. The inventive swatch may be able to both generally lay flat on a flat hard surface and roughly drape over a 3D shape such as a hand or ball, though the link size and swatch flexibility may usually not touch the entire 3D shape.

The present invention uses a sheet of flexible links, herein defined as a swatch. While the applicant uses the term chain mail, the current invention does not use traditional ring mail weaves of wire or sharp-edged loops or large protective garments. Rather the links and the swatch are designed to provide several important physical attributes to maximize enjoyment or satisfaction of a user playing with that swatch.

The swatch of this invention has a very particular type of segmented motion due to link spacing and connection means, unlike a completely flexible cloth or thin silicone elastomer sheet. The direction of motion is constrained in that the swatch bends in more than one direction, but by a nonlinear method. The motion is not necessarily simultaneous in all directions or linear at all swatch conformations. For example, the swatch may simultaneously bend in multiple directions to increase hand contact and variety of contact.

The term swatch as used in this disclosure refers to the chain mail sheet that comprises the links and has the size, and flexibility designed to impart the psychological and physiological effect on the user. It is predominantly defined by X (ML), Y (MB) and Z (MT) dimensions, but to maximize intended psychological benefits, also requires specific combinations of ranges of at least some of: link size, flatness, weight, link periodicity, direction of bend, flexibility, mode of motion, acceleration of motion, sound, and look and feel. These should be addressed in both static conditions lying on the skin and during motion. Flexibility is defined as the ability to bend without excessive force such that at least three non-adjoining links can touch. The swatch comprises 30-200 links, preferably 40-150, and more preferably 40-100 links.

When placed on a flat hard surface, on the X-Y plane, gently pulled out to its maximum area, and then released, the swatch is herein defined to be in its at rest state, and comprises at least a majority and preferably all or almost all of the following attributes:

A) The general swatch shape may have an aspect ratio no greater than an equilateral triangle or otherwise approximate no more than a 1:4 X:Y ratio, preferably less than a 1:1.33 X:Y ratio of the maximum X and Y dimensions. In the Z direction, the shape should give the swatch a look of a roughly approximate sheet when at rest with the totality of the links, not counting voids, averaging two parallel surfaces that encompass the operative upper surface and the operative lower surfaces. In an embodiment the two parallel surfaces may differ.

In an embodiment the swatch may approximate in the X-Y plane the shape of a circle, ellipse, triangle, pentagon, hexagon, octagon, polygon, square, rectangle, rectangle with missing corners, or any combinations thereof. In a preferred embodiment, the swatch shape in the X-Y plane may approximate a triangle or a between square with missing corners and an octagon. In another embodiment the swatch shape in the X-Y plane may be an ellipse. In a particular embodiment the swatch shape in the X-Y plane may approximate a circle.

B) The shape of the link is mainly limited by the loop or other connection means location to determine direction of bend and how the shape of the link and the loop constrain the direction and extent of motion with the adjacent links and therefore the overall swatch and the feel to the skin. In an embodiment shape may be determined by performance to cost, manufacturing, packaging or aesthetics.

C) The swatch's average ML and MB when taken to approximate between an ellipse and a rectangle may have an X-Y aspect ratio within 1:1 to 1:4.

D) When at an at rest state, the swatch X-Y area may be from 25 cm² to 1,000 cm², preferably from 80 cm² to 500 cm², more preferably from 80 cm² to 250 cm², and most preferably from 100 cm² to 200 cm². In an embodiment, the swatch may approximate a square or square with chamfered corners with a length and width within the range of 6 cm to 10 cm. In another embodiment, the swatch may approximate a circle with a diameter from 3 cm to 10 cm.

E) Other than the links themselves, the swatch is highly flexible. At least one edge, and preferably all edges, of the swatch sheet should be able to touch the opposite XY side of the swatch when the swatch is freely bent in any direction from the X-Y plane, over a rigid rod with a diameter of less than 3 mm.

F) The swatch may bend in multiple directions so that it has sufficient drape that it may at least loosely drape over at least one of a Reuleaux tetrahedron, rounded tetrahedron, ellipsoid or hemisphere with a maximum diameter or edge of 5 cm and surface area of less than the swatch XY area. In one embodiment the swatch may be easily crumpled in three dimensions to change from a X-Y sheet to a 3D structure with a Z dimension of at least 3 times that of the sheet's MT. The swatch can be rolled over a dowel with a diameter of at least 2 times the greatest dimension of a link.

G) The swatch is sufficiently loose such that it may bend under its own weight.

H) The swatch may have some give. By give as this term is used here we mean that the width of the gaps formed at the top operative surface or at the bottom operative surface may be increased or reduced by the user because the interconnecting links are loosely connected allowing a link to move in the X or Y direction relative to its neighbouring link. This movement or give in the X-Y plane may result, for example, that the preferred minimum X-Y area may be within 70% to 96% of the at rest area. The at rest area is also referred to herein as a maximum area. The preferred method to measure this void space allowing this movement is volumetric density, defined herein. This difference is preferably sufficiently limited to avoid holes large enough to allow fingers to poke through.

I) The swatch may comprise flat or approximately flat external surfaces available to touch.

J) The swatch should not have greatly uncomfortably sharp edges or cuttingly sharp protrusions, but may comprise an edge between flat and rounded surfaces.

The swatch thickness (MT) is within 3 mm to 10 mm and preferably within 4 mm to 7 mm. The thickness of the swatch (MT) may be sufficiently large for preventing formation of sharp surfaces and for allowing human touching and perception of individual links, but not so large as to impede the intended motion, looseness, and drape of the swatch. A preferred thickness may partially be dependent on the material weight and strength. For example, a stronger material, e.g., a glass or mica filler may provide the required strength for thinner walled loops and, therefore, thinner swatches, and to provide a pleasing weight.

The swatch may be made of at least one type of link, meaning that all the links are of the same shape and size. However, more than one type of links may be used. In an embodiment 2-3 types of links may be used. In a preferred embodiment one type of link is used, though its orientation may differ.

In an embodiment, the swatch may approximate a polygon such as a triangle, an octagon, etc, with a plurality of swatch's corners. In another embodiment, the corners may be eliminated because from an operational perspective they may be too sharp, droopy, loose, or unconstrained.

Each link in the swatch, other than optionally corner links, may be connected to at least three other links. In a preferred embodiment, each corner and edge link may be flexibly connected to 2-4 other links, and each other link may be flexibly connected to 3-6 other links, and preferably a majority of links are connected to 3 other links. The preferred embodiment has 3 connection means per link generally forming a triangle from the flexible connection perspective in a hexagonal overall pattern, while visually the link may approximate other shapes described herein.

In an embodiment, additional links with different flexibility or lack thereof that may provide additional functionality such as fragrance, aroma, LED light, sound, color, and the like, may be employed in addition to the flexible links. Preferably, link types may be less than 9, more preferably two, and most preferably one.

The swatch may have an overall macro roughness that is greater than that of a woven textile fabric but significantly less than a traditional ring chain mail mesh. In other words, the swatch may have an overall macro smoothness that is less than that of a woven textile fabric but significantly more than a traditional ring chain mail mesh. The present invention achieves this by employing a generally flat bumpy external surfaces and a sufficiently large ratio of touch surfaces to the total area covered by the swatch. These touch surfaces combined with a smaller percentage of void space are present in both in a flat X-Y sheet form as well other conformal forms of the swatch.

The swatch may have a flatness wherein 5-90%, preferably 20-90%, more preferably 40-90% and most preferably 60-90% of the touchable surface (also referred to as the contact area of the swatch) of the top side and optionally the top and bottom side is flattened to increase the surface area felt during use. The contact area of the swatch is the area of the interconnected links which come into contact with the skin of a user, for example, the skin of a hand or fingers. In a preferred embodiment, when the swatch is pressed against a flat inkpad with sufficient pressure to ink either the operative upper surface (e.g., 102A) or operative lower surface (e.g., 102B), and then that swatch is pressed against a piece of paper resting on a hard flat surface, or on an adult human arm, the resulting ink on paper image highlights at least 5% of the image area.

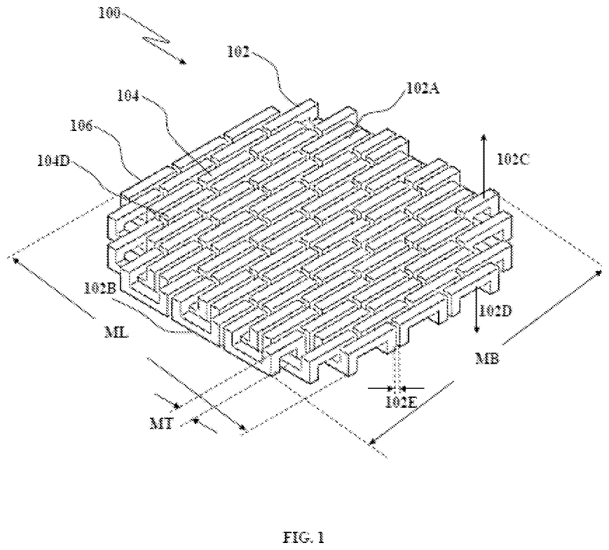

An individual link not connected to other link is not measured because it may have side or internal corners or surfaces not intended to significantly contact the skin in the final swatch. This swatch bumpiness, not to be confused with surface roughness of individual link surfaces, is demonstrated in FIG. 1.

The swatch may have a sufficient solid coverage such that when the swatch rests on a flat surface and in smallest contracted size without continuous external applied force, at least some portion of the swatch covers 20% of the X-Y plane, preferably, at least 25%, and, more preferably, at least 33% of the X-Y plane. This coverage is best measured by placing the swatch at rest on a flat light table and measuring the reduction in transmitted light. Ring mail and other link forms with large extremities but small material cross-section may not provide this coverage.

The volumetric density (in XYZ space) may affect important aspects of the swatch and its use such as but not limited to weight, feel, and looseness. Volumetric density may also be used as a method to measure such properties, but is defined to describe the general swatch and not the link material or material porosity within the link. This measurement compares the weight of a swatch stretched to its maximum normal dimensions as compared to the weight of a solid of the same dimensions, made of the same plastic. For example, a glass filled nylon swatch weighing 87 grams and having a maximum volume of 135 cm³ when compared to a glass filled nylon cube of 135 cm³ weighing 174 grams, would result in a swatch density of roughly 50 vol %. For a swatch made of plastic interconnecting links, the preferred volumetric density when contracted or at rest, is 20 to 70 vol %, preferably 33-60 vol %, and more preferably 40-55 vol %. Some designs that do not stretch significantly in the XY plane, may require more volumetric stretch to ensure sufficient flexibility. The generally high density is driven by a proportionately large link weight which is largely driven by a proportionally large link diameter (LD). The links are not flat traditional rings, but rather are highly bent or incorporate highly bent pillars in order to lay relatively flat with other rings without the protruding perpendicular rings of traditional chainmail, and allowing generally free movement between neighbouring links. In a preferred embodiment, the links have a flattened operative upper surface (e.g., 102A) and operative lower surface (e.g., 102B) to increase contact area with the skin and to reduce unpleasant roughness. This flattening may be anywhere between the flat interface caused by a 3D printer bed to the extended, along their centerline, yet still generally round or elliptical along their cross-sectional profile, of which one example is shown in FIG. 1. A flat surface with sharper transition edges is better suited for stimulation while the extended, rounded surface is better suited for calming. The operative upper surface and operative lower surface do not have to be identical and in one embodiment differ sufficiently to offer two different experiences based on which side is mainly used against the skin.

The swatch consists of one layer of links, though some embodiments may also comprise loose, highly flexible connecting means such as loops, thread, fiber, filament fabric or sheet included in this layer.

The swatch may have an aesthetic look that includes at least one of a colour, a surface printing, a coating, a painting, and a pattern of coloured links. The polymers may be translucent. Some of these treatments such as coating may be multifunctional, e.g., also affect feel and motion. In one embodiment the links are opaque. In another embodiment the links are translucent or transparent.

The swatch may not have rigid frames that impede intended flexibility and drape. The only exception is the embodiment such as a keychain or retail security connection point where the key ring or other external connector means limits motion of its adjacent edge. The swatch may optionally have differing edge links. The swatch may, for example, be generally uniformly flexible along the swatch, with the same type of link or links throughout the swatch, though such links on the edge may naturally have less support from other links and may be more pendent. Although different types of interconnecting links may be used, a preferred link may have sufficient complexity and volumetric coverage. A preferred link may not be a simple circle or loop. A preferred link may have at least twice the contact with a flat surface than does a chain mail made of wire loops. In one embodiment, a fiber passes through at least some of the links. In one embodiment, links are sewed together or attached by small preferably flexible loops. However, these loops may not normally protrude the upper surface or lower surface.

The links are the discrete parts that make up the swatch and also have very specific requirements in order to impart or maximize the beneficial interactions with the user during intended use of this invention.

The maximum dimensions of a link are X or length (LL), Y or, a breadth (LB), and Z or a thickness (LT). The size partially depends on the material density. For plastic and filled plastic, the preferred LL and LB are within 0.3 cm to 2.5 cm. In the preferred embodiment the maximum dimension of a link is within 10-20 mm, more preferably 12-19 mm, even preferably 14-16 mm. For the less preferred embodiment of a square, rectangle, or version thereof without corner links, the preferred LL and LB are within 1.1 cm to 1.5 cm. For triangular, LL (base) and LB (base to apex) are within 1 cm to 2 cm, and preferably within 1.2 cm to 1.7 cm. Preferred link shapes are polygons or ellipses allowing motion in at least 3 directions. Large links, e.g., links having sides >2 cm are not preferred. The diameter of the circle approximating the average cross-section of the link is important in that it determines both the weight of the link and swatch, and it ensures that enough of the swatch contacts the skin. If the links are too large and the link profiles also to narrow, then spaces between the links allow significant intrusion of finger tips which is not preferred. If the link profiles are to narrow, then the swatch would resemble a traditional chainmail made of narrow ring links that feels so abrasive and harsh against the skin that its use required a protective leather jerkin. The present invention requires thick links more akin to jewellery that feel pleasing against the skin at rest, but the present invention also requires a pleasing feel during repeated motion. While the cross-sectional shape or profile of links typically vary as you follow their center line, the most important cross-section to feel are those with potential to touch the skin at rest or during motion. A secondary consideration is that the minimum cross-sectional dimension is most likely to be subject to breakage. For these top and bottom sections of the link, if one approximates their average cross-section to a circle, then the diameter of that circle would have a preferred dimension of 2-4 mm, more preferably 2.5-3.5 mm. The other cross-sectional shapes and dimensions are predominantly designed for link-to-link free motion and link strength.

The link thickness (LT) defines overall maximum thickness (MT) between the upper operative surface and the lower operative surface. LT is proportionate to weight and feel. For plastic LT may be within 3.5 mm to 20 mm, and preferably within 4 mm to 10 mm. An embodiment has an LT of 3-4 mm. LT may not be too thick or it may not feel as pleasant and often may interfere with flex and drape. Metal is not the preferred embodiment due to both cost and to the weight resulting from large, thick links being too heavy to promote rapid movement of the swatch. Foams or densities below 50 w/w % of a thermopolymer are not preferred because they do not provide sufficient weight, even with large, thick links, to promote preferred weight against the skin and preferred rapidity of movement and link acceleration.

The movement area may be defined in the Z direction as LT minus the maximum link thickness or diameter in any XYZ direction of the top minus the maximum link thickness or diameter in any XYZ direction of the bottom operative surface during motion (e.g., a rectangular cross-section may be thickest at 45 degrees). This movement area must allow free motion of the swatch in the intended directions and intended range of motion, though it may create friction, otherwise slow, or stop motion in other directions depending on the swatch conformation at the time.

The link may have a shape which approximates in the X-Y plane a circle, an ellipse, a triangle, a rectangle, a square, a pentagon, a hexagon, an octagon or approximate another polygon. The link may have a shape having an X:Y or ML:MB aspect ratio no greater than 1:2, preferably less than a 1:1.33. While it is rather easy to measure for a rectangle or ellipse, other shapes such as an equilateral triangle for example would also fall within this ratio. Preferably, ML and MB are maximum dimensions. Less preferably, ML and MB are average dimensions. In the Z direction, the shape of the link may provide significant smooth feeling surface to the touch. The top and bottom surfaces of the link may be identical in terms of smoothness, shape, and size, although their orientation may differ as, it is shown, for example in the embodiment of FIG. 1. However, in other embodiments, the top and bottom surfaces of the link may differ not only in orientation but, also, in at least one of smoothness, shape, and size. For example, in an embodiment the bottom surfaces of the link may be substantially flat while the top surfaces may include a flat portion and a curved portion or only a curved rounded portion. Preferably, the shape of the link from a top or bottom view (i.e., viewed perpendicular to the XY plane in which the sheet lies) may be a square, a triangle, or a modified square or triangle having their corners smoothed out. Larger number of connections over 3-4 connections per link and one per loop increase the complexity of the design and are generally not preferred in that they reduce the non-linear motion and more approximate apparel fabric. The connections may preferably be 3 or 4 per link, more preferably 3 connections per link, with 2-3 per link at the swatch edges. The link may be formed to include a plurality of at least one of the following types of elongated elements including beams or rods having a circular, oval, or rounded rectangular cross section. For example, the elongated elements may include a straight cylinder, a bend cylinder, a straight beam, a bend beam, a twisted cylinder, a twisted beam pillars, and other rods that are configured to provide the desired general shape of the link and have at least a portion thereof been flat or substantially flat surface in the XY plane. For example, in an embodiment, the elongated element may have an XY plane surface that is a combination of a flat and a curved surface. In an embodiment, the link may have a Cuban chain link shape significantly modified to connect and flex in a 2-D sheet.

In a preferred embodiment, the largest dimensions of the swatch approximate a shape being or in between a rectangle with missing corner pieces and an ellipse, more preferentially being or between a square with missing corner pieces and a circle. The corner pieces in this embodiment are defined as links only connected to one other link. The shape of the individual links preferably has rounded, thick link members with a high weight to volume ratio similar to links known in the jewellery industry as a Cuban link. While in jewellery Cuban links are typically used in a 1-dimensional (1-D) chain that is only one link wide or sometimes 1-5 links wide, the present invention uses links that have shapes optimized for sheets and are all connected to at least two other links and most of the links are connected to at least three other links. This preferred embodiment consists of roughly identical or similar links that comprise plastic. Unlike the Cuban link used in jewellery, the link design of the present invention is designed to have a larger void space and a large flexibility in both directions from the X-Y plane such that any edge of the swatch when at rest in the X-Y plane can touch any opposite edge of the swatch when the swatch is bent in any plane perpendicular to the X-Y plane.
The shape of the link is mainly limited by the loop location, shape, and orientation that determines bend direction and how the shape of the link and the loop constrain the direction and extent of motion with the adjacent links and therefore the overall swatch. In an embodiment the closed link comprises the loops without additional pillars. Sharp corners are also to be rounded or minimized when they may provide an uncomfortable sensation by the user. In a preferred embodiment imperfections in the plastic parts are acceptable as long as they don't materially scratch the skin. In a preferred embodiment the surface is treated to reduce high aspect ratio surface protrusions or other surface defects. In one embodiment there is a transition edge between a flat bottom operative surface and other curved surfaces.

A Loop is the part of the link that holds an adjoining link and allows generally free motion of that adjoining link in a constrained motion, direction and manner. A link may be merely the space between posts. A loop is defined as the physical or functional method of loosely coupling links. Rigid hinges are not contemplated. Single wire loops or other similar single simple circles or ovals without significant contortion and wall thickness are not contemplated other than in swatches with at least two link types where the smaller circle serves as a connecting link, and that connecting link is generally shielded from the skin in most swatch configurations by the other type, non-circle links. More complex link shapes may contain circle or loop elements such as two at least partially flattened circles connected by posts or loops. A loop may be combination (e.g., pillars in center, loops on outside to retain “loose” feel during motion. Loops may be at least partially coated to modify feel or to apply friction to motion. In the preferred embodiment, there may be only one connection per loop, though the loop may be defined by position or other constraints to motion than a completely closed loop. Concave Curb Cuban link chain and Miami Cuban Link Chain are one-dimensional examples incorporating such a link that contains only one opening but functionally defines 2-6, preferably three, loops. The walls, pillars, freeform enclosure, or other link parts defining the loop may be strong enough to withstand repeated play without application of force beyond the swatch weight.

All or a majority of the X-Y plane areas of the links may have a flat or substantially flat portion of their externally facing surface area of the link's external upper surface and lower surface. Substantially flat includes small deviations from flat due to manufacturing limitations, intentional inclusion of edge chamfer or fillet, and intentional inclusion of smooth protrusions that impart roughness. In a preferred embodiment, both the top side and the bottom side have identical flat or substantially flat surfaces. In an embodiment one of the top or bottom sides comprise non-overlapping plates sufficiently separated to allow the intended range of motion in at least one Z direction.

The links are rigid. For example, rigid links made of a polymer material such as a thermoset or thermoplastic material or less preferably metal may be used for the swatch. In an embodiment, the links may be optionally coated with a metal or polymer coating. For example, the coating may be an elastomeric or a colored coating. The preferred material comprises plastic, preferentially all or mostly a common thermoplastic or co-polymer, preferably selected from the group consisting of ABS, polyester, nylon, or other polymers that are strong enough to resist breaking under normal use. Very soft flexible elastomers or foams are not preferred except as optional interconnecting means as they reduce the desired bumpiness. A filler such as glass beads, glass fiber, mica, silica, or other typical fillers used with thermoplastics or resins are one embodiment. Use of strengthening fillers may be used in a preferred embodiment. The links or swatch may also be surface treated such as tumbling or etching to remove any mold flashing or other high aspect ratio surface protrusions.

Typical thermopolymer material density of the solid part of links themselves are preferred, preferably having a material density of 0.9 g/cm³ to 1.9 g/cm³, more preferably 1.0 g/cm³ to 1.5 g/cm³. Links may be solid or have a porosity of up to 50 vol % as compared to the solid coverage of the swatch, leading to an overall material density of 0.45 g/cm³ to 1.5 g/cm³ for each link. Solid links are preferred for both pleasing weight and resistance to breakage. The preferred mode uses an inexpensive, easily manufacturable material such as a plastic.

The links may not have any very sharp external edges that would be touchable in normal swatch conformations and draped forms that would be too uncomfortable. Any touchable edges may be preferably rounded, with a chamfer, fillet, coating or other softening or removal technique. This does not mean that the swatch may be completely smooth. In a preferred embodiment at part of the link surface has a surface roughness sufficient to be felt by human fingers as not glossy smooth. The interplay between hard surfaces and spaces in between in a range of 3D motion is necessary for this present invention's use. In a preferred embodiment, the links are rounded with flat or substantially flat profile on both external XY plane surfaces. The inside part of the link that is not normally touchable is not important as far as roughness, sharpness and finish other than in how it adversely affects rate of motion or noise of the swatch.

The operative upper (top) surface and operative lower (bottom) surface are connected by at least partially vertical connections between top layer and bottom layer. The number of posts or other such connections may vary between 3 and 13, preferably between 3 and 12. The posts may have varied forms such as but not limited to pillar, curved, loops, free forms connecting top and bottom. Preferably posts comprise a curved component and are not only vertical. This curved post allows smoother flexibility and reduces protrusions of the edges of the links when the swatch is severely bent. Links may have a mix of posts, angled posts, and other types of vertical connections (e.g., curved). In an embodiment there are minimum posts and a thread or fiber may connect the links, but this is less preferred because it does not sufficiently constrain the degrees and range of motion. In an embodiment links may also comprise or simply contain one or more highly bent loops. Most preferably the posts are integral parts of the link.

The terms such as flat and substantially flat surface, includes completely flat and approximately flat surfaces, which are not completely flat due to macro bumps due to the individual links and the exposed link surfaces and rounded edges. An operative upper surface and operative lower surface includes surfaces that are or appear to be upper and lower respectively when in use and link corners that may be exposed when the swatch is bent.

The present invention is now described hereinbelow with reference to the accompanying drawings, wherein FIG. 1 illustrates an isometric view of a chain mail mesh of the presently disclosed invention, wherein the chain mail mesh is in the form of a sheet with identical rounded triangle-shaped interconnecting, polygonal links.

The present invention provides a chain mail mesh 100 which when employed by a human suitably, leads to a significant reduction in stress. Typically, the chain mail mesh 100, when contacted with and/or moved over a body part (such as, for example the skin of the top part of a hand) of the human provides a soothing effect to the body part. Also, the chain mail mesh 100 functions as an anti-fidgeting device that engages the attention of a human and releases its anxiety and stress by the repeated use of the chain mail mesh. The chain mail mesh 100 (FIG. 1) of the present invention is different from those encountered in the prior art. A conventional chain mail mesh having ring-shaped interconnecting links and made of metal. The conventional chain mail mesh has a very bumpy surface and is not at all smooth. By contrast, the chain mail mesh 100 has relatively smooth contact surface(s) imparted by the flat or substantially flat surfaces of the employed links in the external top and bottom XY plane surfaces, which when contacted with and/or moved over a body part (such as the skin of a hand) of the human, has a much higher contact area with such body part and a much smoother and pleasant texture (or feel), thus providing a soothing effect to the body part.

More specifically, in accordance with an aspect of the present invention, a chain mail mesh 100 in the form of a sheet 102 is illustrated in FIG. 1, wherein a plurality of interconnecting, polygonal links 104 are interconnected to define the sheet 102. The chain mail mesh 100 comprises the interconnecting, polygonal links 104 suitably interconnected to define the sheet 102. The sheet 102 has a maximum predetermined length (ML), a maximum predetermined breadth (MB), a predetermined thickness (MT) and a predetermined weight (MW). Each of the interconnecting, polygonal links 104 have at least one degree of freedom of movement in at least one direction with respect to its neighbouring polygonal links and have a maximum length (LL), a maximum breadth (LB), a thickness (LT) (FIG. 1), and weight (LW). Further, each of the interconnecting, polygonal links 104 are interconnected to at least one, preferably three, neighbouring interconnecting polygonal link 104, which combine to form an operative upper surface 102A and an operative lower surface 102B of the sheet 102. The link sections comprising at least a portion of at least one of the operative upper surface 102A and the operative lower surface 102B of the sheet 102 are configured to be rounded along its cross-sectional profile and flat or substantially flat along its center line such that they generally feel smooth, which can produce a calming effect when contacted with or moved over, or combinedly contacted with and moved over a human's body portion which is apt to sense of touch. When viewed from the side the link profile projects a squashed ellipse flattened on its two longest sides rather than a circle. In an embodiment, the interconnecting, polygonal links 104 may be interconnected to the at least one, preferably one, neighbouring interconnecting polygonal link directly. Not all loops have to be utilized. In another embodiment, the interconnecting, polygonal links 104 may be connected to at least one neighbouring interconnecting polygonal link via an intermediate connecting member (not shown in the figures).

In accordance with an embodiment of the present invention, the length (ML) and the breadth (MB) of the sheet 102 may be independently in the range of 50 mm to 250 mm, and the thickness (MT) of the sheet may be in the range of 3 mm to 10 mm, preferably in the range of 4 mm to 7 mm, when the sheet is placed on a substantially flat surface, whereas the length (LL), and the breadth (LB) of the interconnecting, polygonal links may be independently in the range of 2 mm to 25 mm, preferably in the range of 4 mm to 17 mm, and the thickness profile (LP) of the interconnecting, polygonal links is in the range of 2 mm to 4 mm, preferably in the range of 2.5 mm to 3.5 mm.

Further, the weight (MW) of the sheet 102 may be varied. In particular, the weight of the sheet 102 may be varied by employing heavy or light interconnecting, polygonal links 104 and polygonal end cap links 106. In an embodiment, a mix of heavy and light interconnecting, polygonal links 104 may be employed. In another embodiment, all the interconnecting, polygonal links may have the same weight. In yet another embodiment, the weight of the interconnecting, polygonal links and that of the polygonal end cap links may be the same or different. In an embodiment, the links are the same or roughly similar and the weight of the sheet is within 50 g to 100 g.

The (LW) of the interconnecting, polygonal links 104 may be in the range of 0.1 grams to 2 grams. In another embodiment, the weight (LW) of the interconnecting, polygonal links 104 may be in the range of 0.18 grams to 1.8 grams, more preferably 0.4 grams to 1 gram. In one embodiment, the weight (LW) of the interconnecting, polygonal links 104 may be within the range of 0.18 grams and 0.9 grams.

In accordance with an embodiment, the weight (MW) of the sheet 102 may be in the range of 10 grams to 1,000 grams, more preferably 50-200 grams, more preferably 50-100 grams. In another embodiment, the weight (MW) of the sheet 102 may be in the range of 50 grams to 100 grams. In one embodiment, the weight (MW) of the sheet may be within the range of 55 grams to 60 grams.

In accordance with an embodiment of the present invention, the interconnecting, polygonal links 104 may have a rounded triangle shape as depicted in FIG. 1. The interconnecting, polygonal links 104 may comprise at least two lower horizontal connector members (1042, 1044), which define an operative lower surface 104B of the interconnecting, polygonal links 104, and at least two upper horizontal connector members (1046, 1048), which define an operative upper surface 104A of the interconnecting, polygonal links 104. Further, the at least two lower horizontal connector members (1042, 1044) are connected to the at least two upper horizontal connector members (1046, 1048) at corner posts (1050) to define a continuous link.

In accordance with another embodiment of the present invention, the interconnecting, polygonal links 104 may have a rounded triangle shape as depicted FIG. 1, wherein, the interconnecting, polygonal links 104 comprises at least two lower horizontal connector members (1042, 1044), which define an operative lower surface 104B of the interconnecting, polygonal links 104, and at least two upper horizontal connector members (1046, 1048), which define an operative upper surface 104A of the interconnecting, polygonal links 104. Further, the at least two lower horizontal connector members (1042, 1044) are connected to the at least two upper horizontal connector members (1046, 1048) at corner posts (1050) to define an incessant link, and a lower transverse intermediate connector member 1052 is disposed between and connected to the at least two lower horizontal connector members (1042, 1044) and an upper transverse intermediate connector member 1054 is disposed between and connected to the at least two upper horizontal connector members (1046, 1048). The various corners and edges in the illustrated embodiment are shown as forming 30-90 degree angles, however, in an embodiment the corners and edges may comprise a fillet or chamfer to improve the overall smoothness of the swatch.

The provision of the lower transverse intermediate connector member 1052 and the upper transverse intermediate connector member 1054 may further increase the surface area and also the smoothness of the operative upper surface 104A and the operative lower surface 104B of the interconnecting, polygonal links 104 and that of the sheet 102 and at the same time may provide strength to the interconnecting, polygonal links 104.

The swatch may include a macro-smoothness which refers to the texture of the swatch as it feels to the user while the swatch is moved over the skin of the user and a micro smoothness which refers to the smooth texture of the touchable surface of the individual links which are free of sharp protrusions or 1-D corners. In an embodiment, the touchable surface of the links although not mirror smooth may not have protrusions from the bulk of the link of greater than 0.1 mm, with any another dimension within 75 degrees of surface of the link of less than 0.1 mm. The macro-smoothness, in addition to the smoothness of the touchable surface of the links, depends on the shape of the links, their number per area and also on the gaps between the links.

In accordance with the present invention, the interconnecting, polygonal links 104 (FIG. 1 may have the operative upper surface 104A, the operative lower surface 104B, and one or more substantially smooth edges 104C. The operative upper surface 104A, the operative lower surface 104B and the one or more substantially smooth edges 104C may have a shape which may be selected from the group consisting of flat or planar, concave, convex, and any combinations thereof. Any other shape is also well within the ambit of the present invention provided the shape provides a smooth or generally pleasing feel. In an embodiment, the operative upper surface 104A and the operative lower surface 104B of the interconnection polygonal links each have a flat surface.

In accordance with an embodiment of the present invention, at least one layer of polygonal end cap link 106 is connected to an outer most layer 104D of the interconnecting, polygonal links 104 of the sheet 102. In an embodiment, the polygonal end cap links 106 may be the same as that of the interconnecting, polygonal links 104. In another embodiment, the polygonal end cap links 106 are different from that of the interconnecting, polygonal links 104. In yet another embodiment, at least some of the polygonal end cap links 106 may be the same as that of the interconnecting, polygonal links 104 and some of the polygonal end cap links 106 are different from that of the interconnecting, polygonal links 104 in a given sheet 102. In the present context, the meaning of different or same polygonal end cap links 106 is that the structure or design or form of the polygonal end cap links 106 is same as that of the interconnecting, polygonal links 104 or the structure or design or form is different as compared to that of the interconnecting, polygonal links 104. Any other shape of the polygonal end cap links 106 may include a ring or a continuous loop-like structure. In the present context, the meaning of different or same polygonal end cap links 106 may also mean that the composition of the interconnecting, polygonal links 104 or the material of make of the interconnecting, polygonal links 104 may be different or same.

In the preferred embodiment, the swatch should be able to freely bend from an at-rest stage in the X-Y plane. The flexibility should be sufficient to bend with an average radius of curvature of at least 5 cm, or measurable from the at-rest stage to generally circumvent a dowel with a diameter of at least 10 cm and more preferably at least 5 cm, when the cylindrical edge of the dowel is placed in contact in any direction of the X-Y-plane of the swatch. Naturally, edge of the links may protrude from the general swatch surface during this bending, but in the preferred embodiment these increased protrusions reduce the average maximum thickness of the swatch by less than 50%.

In accordance with an embodiment of the present invention, the interconnecting, polygonal links 104 may be in the form of polygons, wherein the polygon is at least one selected from the group consisting of a three-sided polygon, a four-sided polygon, a five-sided polygon, a six-sided polygon or a higher order polygon. The shape of the interconnecting, polygonal links 104 may be at least one shape selected from the group consisting of a polygon, a circle, an ellipse, a hexagon and any combinations thereof. In an embodiment, the sheet 102 may comprise a mix of links with some interconnecting, polygonal links 104 being four-sided polygons, and some others being six-sided polygons. In another embodiment, the sheet 102 may comprise interconnecting, polygonal links 104 which are a mix of three-sided and four-sided polygons. In an embodiment, the interconnecting, polygonal links 104 may have a rounded triangular overall shape and three interconnecting neighbouring links when viewed from the top.

The interconnecting, polygonal links 104 and the polygonal end cap links 106 may be each independently manufactured from a material selected from the group consisting of plastic, thermoplastic, metal, and any combinations thereof. The choice of the material depends on the drape of the sheet 102 to be achieved, the body portion over which the sheet 102 is to be used, and the weight of the sheet 102 to be achieved. In an embodiment, the interconnecting, polygonal links 104 and the polygonal end cap links 106 are each independently manufactured from plastic, preferably a thermoplastic material such as a polyolefin thermoplastic including but not limited to homopolymer polyolefins, random copolymer polyolefins and impact copolymer polyolefins. The plastic material may be colored or alternatively a paint coating may be employed. The plastic material may be metal coated. The drape, the weight, the shape, and the structure and design of the interconnecting, polygonal links 104 are selected so as to provide a smooth feel when contacted with the body portion and provide stress relief when played or engaged with the same. In an embodiment, the interconnecting, polygonal links 104 and the polygonal end cap links 106 are each independently manufactured from plastic. In an embodiment, the interconnecting, polygonal links 104 and the polygonal end cap links 106 are each independently made of thermoplastic material.

In a specific embodiment, the chain mail mesh 100 may include a plurality interconnecting, polygonal links 102 suitably interconnected to define a sheet 102 having a length (ML), a breadth (MB), a thickness (MT), and weight (MW), each of the interconnecting polygonal link 104 comprising at least two lower horizontal connector members (1042, 1044), at least two upper horizontal connector members (1046, 1048), at least one lower transverse intermediate connector member 1052 disposed between and connected to the at least two lower horizontal connector members (1042, 1044), and at least one upper transverse intermediate connector member 1054 disposed between and connected to the at least two upper horizontal connector members (1046, 1048), wherein the at least two lower horizontal connector members (1042, 1044) along with the at least one transverse intermediate connector member 1052 define an operative lower surface 1056 of the interconnection polygonal link 104, wherein the at least two upper horizontal connector members (1046, 1048) along with the at least one transverse intermediate connector member 1054 define an operative upper surface 1058 of the interconnection polygonal link 104, wherein the at least two lower horizontal connector members (1042, 1044) being connected to the at least two upper horizontal connector members (1046, 1048) at corner posts 1050 to define an incessant link, wherein each of the interconnecting polygonal link 104 is made of thermoplastic material and having a length and a breadth in the range of 10 mm to 14 mm, independently, a substantially rounded, polygonal shape, each of the interconnecting, polygonal links being similar in structure, dimensions, and shape, wherein each of the interconnecting polygonal link 104, when not at an edge of the swatch, is interconnected to preferably three neighbouring interconnecting polygonal link 104 combine to form an operative upper surface 102A and an operative lower surface 102B of the sheet 102, wherein the number of interconnecting, polygonal links 104 being in the range of 5 to 15 along the length and the width of the sheet 102 independently, wherein the sheet 102 is either having a substantially rectangular configuration or a substantially square configuration wherein the sheet 102 is laid on a flat surface, wherein at least a portion of at least one of the operative upper surface 102A and the operative lower surface 102B of the sheet generally feels smooth, which produce a calming effect when contacted with or moved over, or when contacted with and moved over a human's body portion which being apt to sense of touch, and wherein the sheet 102 includes at least one layer of polygonal end cap link 106 connected to an outer most layer of the interconnecting polygonal link 104 of the sheet 102, the polygonal end cap link 106 being same as that of the interconnecting polygonal link 104 or the polygonal end cap link 106 being different from that of the interconnecting polygonal link 104.

In an embodiment, at least a portion of the sheet 102 is bendable in at least one direction selected from an operative upward direction 102C of the operative upper surface 102A of the sheet 102, an operative downward direction 102D of the operative lower surface 102B of the sheet 102 and a combination thereof. In particular, the sheet 102 on a macro scale on the size order of the swatch, has a predetermined drape coefficient similar to a cloth or a fabric, but at the same time, on a micro scale on the size order of the links, the sheet 102 has a predetermined radius of curvature when the sheet 102 is placed on a support. This, in turn, means that the sheet 102 though bendable in operative upper and lower directions, is not completely flexible as a cloth or fabric. In an embodiment, the sheet 102 has a predetermined drape coefficient. In accordance with an embodiment of the present invention, the drape coefficient of the sheet 102 may be the same along the length (ML) and the breadth (MB) of the sheet 102. In another embodiment, the drape coefficient of the sheet 102 may be different along the length (ML) and the breadth (MB) of the sheet 102. Further, the thickness (MT) of the sheet 102, when the sheet is in draped configuration, is different from the thickness (MT) of the sheet, when the sheet 102 is placed on a substantially flat surface.

Owing to the above, the bendability and the radius of curvature of the sheet 102, the sheet 102 drapes when contacted with or moved over, or when contacted with and moved over a human's body portion and substantially conforms to the shape of the body portion, wherein the operative lower surface 102B of the sheet 102 in contact with the body portion provides a calming effect thereto.

In order to facilitate the draping of the sheet 102, the interconnecting, polygonal links 104 within the sheet 102 are spaced apart with a gap 102E in an at rest state therebetween, the gap 102E being in the range of 0.2 mm to 15 mm, more preferably in the range of 0.5 mm to 12 mm, thereby configuring a loose hinge between two consecutive interconnecting, polygonal links 104, yet being smaller than the diameter of a finger. A minimum, smaller gap of 0.1 mm, preferably of 0.2 mm is envisioned to avoid pinching or catching of hair. The gap 102E provides the necessary flexibility and hence bendability and the drape to the sheet 102. This gap is measured at rest by transmission through the sheet when placed on a light table, and at maximum such as during skew, is measured with callipers. At the same time, the shape of the interconnecting, polygonal links 104 along with the dimensions and the gap 102E also make the sheet 102 resistant to drape further than a predetermined curvature. It has been discovered by the Applicant that the sheet 102 having the above configuration when contacted with a body portion of a human provides a calming effect thereto. Further, owing to the gaps, and the dimensions of the interconnecting, polygonal links 104, at least a portion of the sheet 102 is skewable in at least one direction substantially parallel to a plane of the sheet 102. In the preferred embodiment the XY area of the gaps is smaller than the solid XY area of the link 104, when the sheet is in a configuration having its smallest XY area.

In an embodiment, the dimensions, the shape, the material of make of the sheet 102 are chosen such that the sheet 102 when displaced is capable of generating an acoustic spectrum during typical play or movement between the fingers or two human hands. In an embodiment, the acoustic spectrum has at least one frequency in the range of 20 Hz to 20,000 Hz.

The chain mail mesh 100, the interconnecting, polygonal links 104 and the polygonal end cap links 106 may be manufactured by any known method in the art. For example, the chain mail mesh 100, the interconnecting, polygonal links 104 and the polygonal end cap links 106 may be manufactured by at least one method selected from the group consisting of 3D printing, injection molding (for example, continuous plastic injection molding), snap fitting, casting, such as zinc casting, and any combinations thereof. In an example, the interconnecting, polygonal links may be injection molded separately and then snap-fitted to configure the sheet 102.

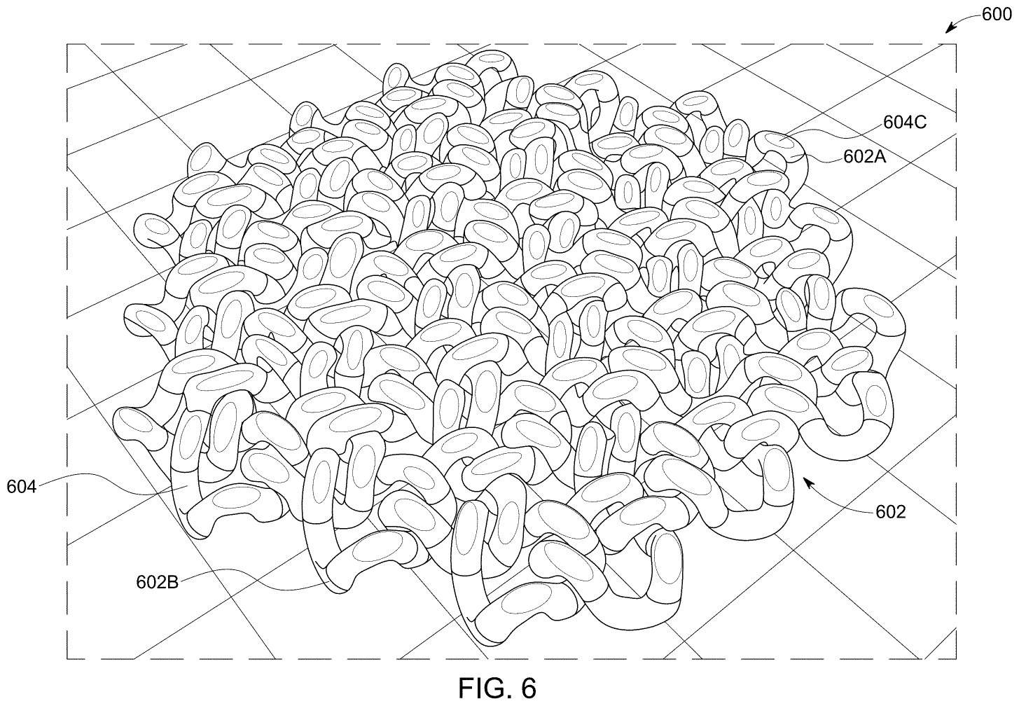

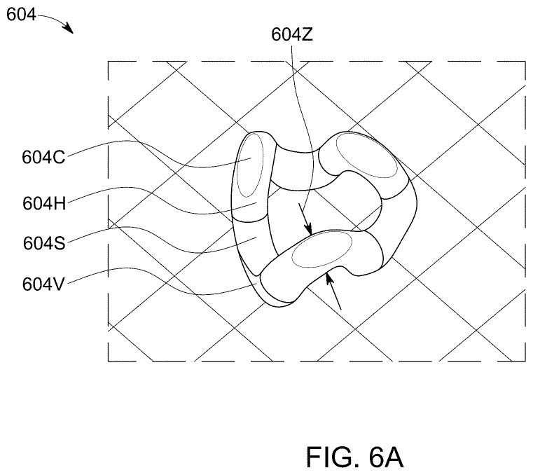

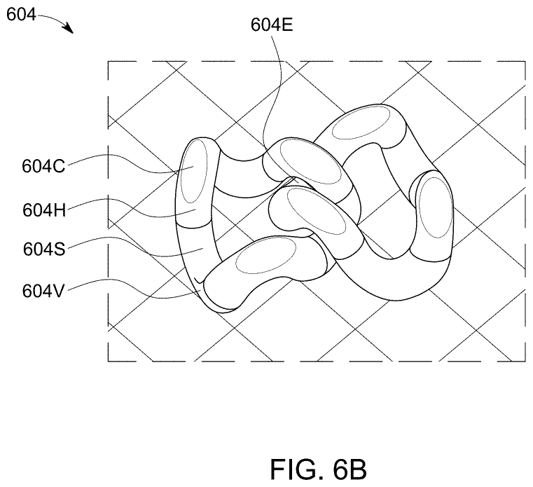

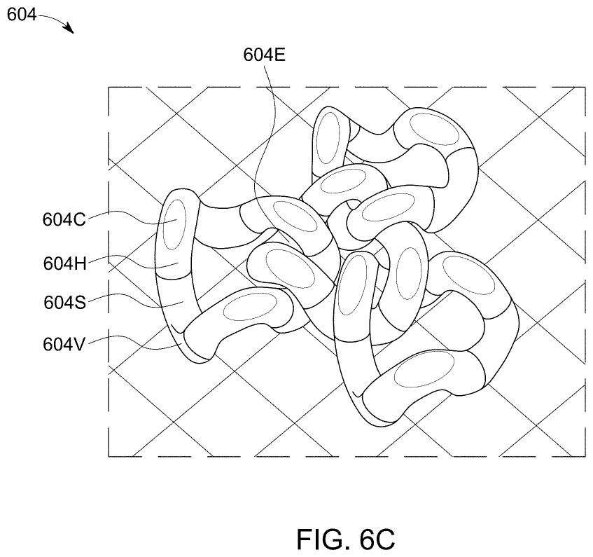

Referring now to FIGS. 6, 6A, 6B, and 6C a chain mail mesh 600 according to an embodiment of the present invention is provided wherein the chain mail mesh 600 comprises a plurality of links 604 wherein each link 604 is coupled to three other links except some of the outermost links 604p each of which is coupled to two other links. Each link 604 comprises three valleys 604V and three hills 604H and may have a generally triangular shape or oval shape when viewed from the top or bottom. The link may be preferably formed of a single, continuous, rod or substantially solid pipe having a generally circular or oval cross-section of a constant diameter. A profile diameter 604Z (or cross-sectional diameter) may be substantially the same along the whole link 604. The embodiment illustrated in FIGS. 6, 6A, 6B, and 6C employs links, each having three hills an three valleys, however, in other embodiments which are variations of this embodiment links having four, five, or six links may be used.

Each valley 604V and each hill 604H may have a generally curved shape. The plurality of the interconnected links 604 may form a smooth operative upper surface, e.g., 602A and a smooth operative lower surface, e.g., 602B which come into contact with the skin of the human and provide a pleasant smoothness to the touch of the human user. Hence, the operative upper surface 602A as this term is used herein refers to the total upper surface of the chain mail sheet that is formed by the plurality of the top surfaces of the interconnecting links 604. Also, the operative bottom surface 602B as this term is used herein refers to the total bottom surface of the chain mail sheet that is formed by the plurality of the bottom surfaces of the interconnecting links 604.

Each hill 604H may have at least one central region 604C that has a flat or substantially flat surface. The plurality of the central regions 604C of the hills 604H when considered together form the operative upper surface 602A of the chain ail mesh sheet. Each valley 604V may also have at least one central region 604C that has a flat or substantially flat surface. The plurality of the central regions 604C of the valleys 604V when considered together form the operative bottom surface 602B. In an embodiment (NOT SHOWN), each valley 604V and each hill 604H may only have a generally curved surface throughout.

Each valley 604V may be continuously coupled via side sections 604s (also referred to as the intermediate section) of the link 604 with adjacent hills 604H. The side section 604s may be a rod or pipe of the same or substantially the same cross-section as the rod or pipe that makes the hills 604V and valleys 604H of the links 604. Each link 604 is coupled to another link 604 as shown in FIG. 6B allowing a gap 604E between the links. Too small of a gap does not allow free draping and motion for the chain mail mesh. Too large of a gap allows fingers to get caught between the links, and also do not provide enough percentage of contact areas with the skin per swatch (too much space or air versus the contact plastic surface of the sheet). In an embodiment, the gap may range between 01 mm and 0.4 mm. Also, the maximum inner dimension of the space outlined by each link cannot be too large because then fingers can be caught inside each of the links.

In accordance with another aspect of the invention, a method of reducing stress or otherwise improving a mental condition or the symptoms of a mental condition in humans employing the chain mail mesh 100 or 600 hereinabove is disclosed. The method comprises the steps of contacting with or moving over, or combinedly contacting with and moving over a human's body portion which being apt to sense of touch, the chain mail mesh 100, 600 wherein the chain mail mesh 100, 600 intermittently contacts with nerve endings of the body portion thereby provide a calming effect thereto.

In accordance with yet another aspect, a process for reducing stress in humans is disclosed, wherein the process comprises the steps of holding the chain mail mesh 100, 600 in hands of a human, contacting the chain mail mesh 100, 600 with a body portion of the human and/or moving the chain mail mesh 100, 600 over the body portion of the human such that the operative lower surface 102B, 602B or the operative upper surface 102A, 602A contacts with nerve endings of the body portion and thereby provide a calming effect thereto, and repeating the above steps more than once to reduce the stress in humans.

In accordance with the present invention, the body portion with which the chain mail mesh 100 is contacted may be at least one selected from the group consisting of hand, wrist, head, forehead, face, shoulder, back, chest, neck, finger, calf, knee, thigh, hip, waist, foot, heel, ankle, buttock, leg, skin, and stomach, and is not limited to these. The preferred boy part comprises fingers and at least part of one or two hands.

Though the present invention has been described with reference to contacting with and/or moving over, the chain mail mesh, the body part of a human, there may be other ways that may be employed to engage with the chain mail mesh. For example, the chain mail mesh of the present invention may be simply draped over the hand(s), forearm, leg, or foot. The chain mail mesh may be rattled to emit a noise/sound, which may be stress relieving for some. Further, the chain mail mesh may be rubbed against skin or wrapped on a finger or may be placed on a hard surface and rest a body part or skin over it or may be played with by placing it in a pocket.

Further, the surfaces, the operative upper surface and the lower operative surface of the interconnecting, polygonal links and that of the sheet 102, 602 may be made somewhat rough rather than being smooth as described hereinabove, which may be used for playing with.

The present invention may also be used to better observe signs of stress and other underlying feelings during psychological therapy sessions, interrogations, job interviews and the like. Applicant has observed that subjects holding the present invention tend to play with it more frequently and with more intensity during more stressful moments. The visual and audible indicators are often somewhat proportional to stress, anxiety or similar feelings, especially when correlated with the same subject.

The present invention is now described with reference to the following examples which are provided to illustrate the present invention more clearly and not to limit the scope thereof.

Manufacturing method—The swatch may be made by any suitable methods including but not limited to 3D printing, UV resin printing, injection molding, zinc casting, and co-moulding. For example, parts may be printed together to save costs. The preferred manufacturing methods are additive manufacturing or injection molding. Parts may be snapped together or better fused in place to minimize choking hazard of links. While the main links are rigid, co-molding with an elastomer or other flexible or soft to the touch material may be employed according to an embodiment. In an embodiment, the links may be foamed or otherwise below theoretical density of their material. Secondary manufacturing treatment may include but is not limited to coating, finishing, partial finishing, grinding, polishing, solvent treatment, etch, flash removal, rounding of sharp edges, thinning, and separating bound links. Manufacturing does not include metal welded rings other than optionally as a connector for the links defined herein. In a preferred embodiment, the links and swatch comprise essentially only one polymer, co-polymer or resin capable of being 3D printed or molded, is essentially rigid, and has a Shore hardness (D) of at least 58.

### Example 1

Hexagonal links are 3D printed from glass filled nylon, or other typical commercial polymer such as PLA or ABS. The links can be called 12 sided polygons if the sloping connector members acting as “pillars” are also included as sides. In one embodiment, the links are printed from translucent UV-curable SLA 3D printing resin. Ideally, the links are printed together in the swatch without any removeable supports used for manufacturing purposes. Harder polymers such as PLA will make more noise than softer polymers such as ABS. The swatch in this example contains 40-100 links. When at rest, the swatch has a 2D shape in the X-Y plane that approximates between a circle and a square. A preferred embodiment has a hexagonal overall link arrangement or bending pattern. The shape of each of the individual links in the X-Y plane is approximate a triangle with rounded corners. In the Z direction, the link undulates such that internal links (i.e., links which are not positioned at an edge of the swatch) are connected to three other links. The purpose is to provide a similar feeling regardless of the X-Y orientation of the swatch. The connecting links may be inverted to better connect to neighbouring links. A link profile diameter range of 2-5 mm, with an average link profile diameter of 2.5 mm to 3.5 mm is preferred to provide sufficient weight, and secondarily to reduce potential of breakage. One or both operative upper or operative lower surface area of the interconnecting, polygonal links 104, 604 may be flattened to increase stimulation, while a rounded surface promotes calming. The other surfaces are preferably rounded to ensure rapid movement of the links with regard to each other. EA preferred embodiment of this example are illustrated in FIG. 6.

The present invention has been described with reference to the specific embodiments and drawings, which are meant for the purpose of describing the invention and its features and not to limit the scope of the present invention. It may be obvious that the invention may be varied in numerous ways. Such variants should not be regarded as a departure from the essence and scope of the present invention, and all such modifications as would be obvious to one skilled in the art are intended to be included within the scope of the present inventions and claims appended herein. While a very specific link is shown, it is merely one illustrative embodiment of the present invention.

1. A method of utilizing a device to diagnose, observe, or modify a mental condition or symptoms of the mental condition, the method comprising: contacting the device with a skin area of the human,
wherein the device comprises: a flexible swatch comprising a plurality of discrete, flexibly connected interconnecting links,
wherein the swatch forms a sheet having a maximum at rest XY area from 80 cm² to 250 cm², wherein the swatch has a weight between 50 grams to 200 grams,
wherein the swatch has a thickness (MT) within 4 mm to 6 mm,
wherein the swatch when extended to its maximum dimensions has a volumetric density of 33-60 vol % as compared to a solid cube of the same maximum dimensions,
wherein the swatch thickness does not increase by more than 50% when the swatch is draped over a cylinder with a diameter of at least 10 cm, wherein the swatch comprises 40 to 100 interconnecting links, wherein the links comprise at least 60 vol % plastic and have a material density of 0.5 g/cm³ to 1.9 g/cm³,
wherein the links can move with relation to each other, wherein the swatch does not materially abrade the skin when moved over the skin solely under its own weight.

2. A method according to claim 1, further comprising first placing the device over a skin area of the human and moving the device over the skin area of the human while at least one portion of the device is in contact with the skin area of the human, and wherein the swatch forms a sheet having a maximum at rest XY area from 100 cm² to 200 cm²,
wherein the links are flexibly connected in a pattern to form the swatch that allows the swatch to flex in 6 Z plane directions perpendicularly relative to an at-rest state in the X-Y plane,
wherein the swatch has a weight between 50 grams to 100 grams, and wherein the volumetric density is 40-55 vol % as compared to a solid cube of the same maximum dimensions, the material density of each link is 1.0 to 1.5 g/cm3, the profile diameter of each link varies between 2.5 mm and 3.5 mm, and the sheet thickness (MT) does not increase by more than 50% when the sheet is bent with a radius of curvature of at least 5 cm.

3. The method according to claim 1,
wherein the method comprises observing the use of the device by the human as a tool for diagnosis or treatment of the mental condition or of symptoms of the mental condition, and
wherein the mental condition or symptoms of the mental condition comprise at least one of:
fidgeting, stress, post-traumatic stress disorder (PTSD), trauma, attention deficit disorder (ADD), attention deficit hyperactivity disorder (ADHD), autism spectrum, distraction, annoyance, lying, general anxiety, situational anxiety, depression, motor function, touch sensitivity, isolating psychiatric conditions, trauma, and the like.

4. The method according to claim 1, wherein the method comprises observing the use of the device by the human as a tool for diagnosis or treatment of a mental condition or symptoms of a mental state or condition comprising at least one of: lying, therapy, preparing for therapy, coming down from psychotherapy efforts to change thinking, preparing mental state, diagnosing stress, diagnosing sources of anxiety, diagnosing depression, treating stress, treating anxiety, treating depression, treating effects of trauma, and the like.

5. The method according to claim 1, wherein the method comprises observing the use of the device by the human as a tool for:
i) diagnosis of mental condition or symptoms of mental condition including lying, stress, anxiety, depression and the like,
ii) diagnosis of at least one reason for the diagnosed lying, stress, anxiety, depression and the like,
iii) for treating the mental condition or symptoms of the mental condition; and
iv) determining the efficacy of the treatment followed by the human for the therapy of the diagnosed mental condition or symptoms of the mental condition.

6. The method according to claim 5, wherein the diagnosis (i) or (ii) or the determination of the efficacy of the treatment (iv) includes observing use of the device by the human at a predetermined interval following the treatment.

7. The method according to claim 5, wherein the diagnosis (i) or (ii) or the determination of the efficacy of the treatments (iv) includes:
asking the human to repeat a pattern of use of the device during an observation session having a predetermined observation time
monitoring via a camera of the use of the device by the human,
recording the video, and
analyzing the video by a professional including a medical doctor, a psychiatrist, a physiotherapist and the like,
wherein said analysis includes recording the number of the repetitions performed by the human within the observation time.

8. The method according to claim 7, further comprising a plurality of observation sessions for diagnosis (i) or (ii), or for determining the efficacy of the other treatments (iv).

9. The method according to claim 7, further comprising a plurality of treating sessions for treating the at least one diagnosed stress, anxiety or depression via us e of the device by the human.

10. The method according to claim 9, further comprising during at least some of the said plurality of treating sessions a professional including a medical doctor, a psychiatrist, a physiotherapist and the like observing the use of the device by the human and collecting use parameters including the number of repetitions performed by the human of a particular pattern of use prescribed by the professional during an observation time wherein the observation time may be the same or less than a treating time.

11. The method according to claim 1, wherein the method comprises:
placing the device on a body portion of the human which is apt to sense of touch, and
moving the device over the body portion,
wherein said device intermittently contacts skin areas near nerve endings of said body portion thereby providing a calming effect to the human.

12. The method according to claim 1, wherein the method comprises:
placing the device on a body portion of the human which is apt to sense of touch,
moving the device over the body portion,
a professional including a medical doctor, a psychiatrist, a physiotherapist and the like observing the use of the device by the human to collect use parameters including the frequency of the use, and
determining based on the collected use parameters at least one of the following:
i) whether the human is lying, or is suffering from at least one of stress, anxiety, and depression,
ii) whether a prescribed treatment for the at least one of the stress, the anxiety and the depression is reducing the stress, the anxiety or the depression,
iii) whether a repeated use of the device by the human reduces at least one of the stress, anxiety, and depression of the human, and
wherein the device intermittently contacts skin areas near nerve endings of said body portion thereby providing a calming effect to the human,
wherein the X-Y shape of the swatch approximates a shape between a rectangle and a circle,
wherein the shape of the link comprises both curved and flattened surfaces, wherein the shape of the link is adapted to provide a slightly rough external surface,
wherein each link has a maximum XY area from 1 cm2 to 5 cm2, a maximum Z height from 5 mm to 10 mm, and an average post thickness from 2 mm to 4 mm,
wherein each link has a ratio of maximum X:Y dimensions of 1:1 to 1:1.5,
wherein the swatch comprises essentially similar links,
wherein every link is attached to 2 to 6 other links,
wherein the majority of links are attached to 6 other links,
wherein the links comprise plastic and
the swatch does not have any touchable protrusions that are sharp enough to reduce mental state of the human.

13. The method according to claim 1, wherein the swatch includes an operative lower surface and an operative upper surface which are substantially identical, the swatch has a repeating pattern with a changeable gap between the links, the gap being from 0.1 mm to 0.4 mm when at rest.

14. A device for improving a mental state in a human, the device comprising:
a chain mail mesh comprising a plurality of links suitably interconnected to form a sheet, each of said links having at least one degree of freedom of movement in at least one direction with respect to its neighbouring links,
wherein each of said interconnecting links is interconnected to at least one neighbouring interconnecting link, and
wherein said plurality of interconnecting links combine to form an operative upper surface and an operative lower surface of said sheet,
wherein the sheet when extended to its maximum dimensions has a volumetric density of 33-60 vol % as compared to a solid cube of the same maximum dimensions,
wherein corners of the links do not protrude by more than 50% when draped over a cylinder with a diameter of at least 10 cm, and wherein at least a portion of at least one of said operative upper surface and said operative lower surface of said sheet is sufficiently smooth during hand motion and of a repeating pattern of the links such than when moved over a body part of the human being apt to sense of touch, induces repeated use or produces a calming effect.

15. The device of claim 14,
wherein the sheet has a length of 50 mm to 250 mm, a breadth of 50 mm to 250 mm and a thickness of 3.0 mm to 8.0 mm,
wherein each link is made of a body of plastic or filled-plastic material having a substantially constant cross-sectional area of a rod or pipe shape having a profile diameter of 2.5 mm to 3.5 mm, and
wherein each link other than an external link is movably coupled with three other links to form the sheet, and
wherein the at least portion of at the least one of said operative upper surface and said operative lower surface of said sheet has an RMS smoothness of 60 to 200.

16. The device of claim 14
wherein the chain mail mesh comprises a continuous body of a rod or essentially solid pipe of a plastic material,
wherein the rod or pipe forms three to six valleys and three to six hills spaced apart from each other at a regular interval in a closed loop configuration,
wherein each valley is followed by a hill and each hill is followed by a valley,
wherein between each valley and an adjacent hill the body of the rod or pipe forms a slanted intermediate element connecting the two together in a continuous manner.

17. The device of claim 16,
wherein the closed loop configuration is approximately circular, oval, or triangular in shape from a top or bottom view,
wherein each hill and each valley has an external surface defined as the surface that comes into contact with the skin of the human when the device is used by the human as fidget device, as a toy, and/or for the modification, diagnosis, or treatment of a mental condition or mental state or symptoms of a mental condition or mental state including anxiety, stress, and the like,
wherein the external surface has an RMS smoothness of 20 to 600.

18. The device of claim 17, wherein the external surface includes at least one central portion that is substantially flat,
wherein each link is coupled to three or four other links except for the outmost links each of which is coupled to only two other links,
wherein a gap between two coupled links is between 0.1 mm to 0.4 mm,
wherein the sheet has a weight of 50 to 200 grams, and
wherein each link has three hills and three valleys.

19. A method of using the device according to claim 14 as a fidget toy for reducing fidgeting, the method including at least one of (i) the user moving the chain mail mesh from hand to hand, (ii) placing the chain mail mesh over a body portion with one or both hands, and/or (iii) moving the chain mail mesh over a body portion
wherein the method focuses undesirable fidgeting by the user to the device and the user feels better or less stressed during this method.

20. The method according to claim 19,
wherein the body portion is a first hand of the human, and the human is holding the chain mail mesh with a second hand and moving the chain mail mesh while at least one of a lower operative surface or an upper operative surface of the chain mail mesh is in contact with a skin portion of the first hand and stimulates nerve endings of said skin portion thereby inducing the human to use the device and to change rate of movement or frequency or both of the motion of the chain mail.

20. (canceled)
